(19) United States
(12) Patent Application Publication
(10) Pub. No.: US 2009/0221589 A1
(43) Pub. Date: Sep. 3, 2009

(54) Use of aminoalcohol derivatives for the treatment of overactive bladder

(76) Inventors: Thomas Trieselmann, Warthausen (DE); Bradford S. Hamilton, Biberach (DE); Stephan G. Mueller, Warthausen (DE); Dirk Stenkamp, Biberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(21) Appl. No.: 12/097,931

(22) PCT Filed: Dec. 18, 2006
(86) PCT No.: PCT/EP06/69856

§ 371 Date: Jul. 29, 2008

(30) Foreign Application Priority Data
Dec. 19, 2005 (EP) 05027738.3
Jan. 26, 2006 (DE) 102006003697.2

(51) Int. Cl. A61K 31/4164; A61K 31/502; A61K 31/4439; A61K 31/422; A61K 31/4178; A61K 31/4184; A61K 31/4196; A61P 13/10

(52) U.S. Cl. 514/248; 514/399; 514/392; 514/343; 514/378; 514/397; 514/394; 514/383

(57) ABSTRACT

The present invention relates to the use of new beta-agonists of general formula (Ia) or (Ib) wherein the groups R¹ to R¹² and R¹ to R⁷, respectively, have the meanings given in the claims and specification, the tautomers, the enantiomers, the diastereomers, the mixtures thereof, the prodrugs thereof and the salts thereof, particularly the physiologically acceptable salts thereof with inorganic or organic acids or bases, for preparing a medicament for the treatment of overactive bladder.

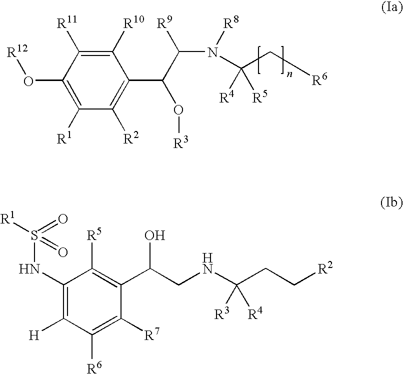

The present invention relates to the use of beta-agonists of general formula (Ia)

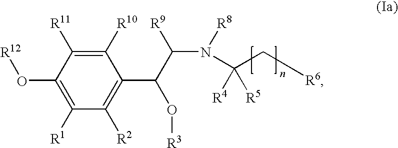

wherein the groups R¹ to R¹² have the meanings given in the claims and specification, and the isomers thereof,
as well as compounds of general formula (Ib)

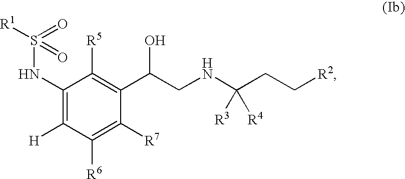

wherein the groups R¹ to R⁷ have the meanings given in the claims and specification, the tautomers, the enantiomers, the diastereomers, the mixtures, the prodrugs thereof and the salts thereof, particularly the physiologically acceptable salts thereof with inorganic or organic acids or bases, for preparing pharmaceutical compositions for treating particular illnesses.

### BACKGROUND TO THE INVENTION

The present invention relates to the use of beta-3 receptor agonists according to WO 2004/039784 for preparing pharmaceutical compositions for the treatment of hyperactive bladder and prostate problems.

Furthermore, the present invention relates to the new use of selective beta-3 agonists according to WO 05/108373 for the preparation of pharmaceutical compositions for the treatment of hyperactive bladder and/or prostate problems.

### DETAILED DESCRIPTION OF THE INVENTION

It has been found that compounds of general formula (Ia) and (Ib) wherein the groups R¹ to R¹² and R¹ to R⁷, respectively, have the meanings indicated hereinafter, may be used for the treatment of urological diseases.

In the above-mentioned general formula (Ia)

R¹, R², R¹⁰, R¹¹ independently of one another denote a group selected from among hydrogen, halogen, CN, NO₂, and —NHCXNH₂ or a group selected from among optionally substituted —COR⁷, —COOR⁷, —CONR⁷R¹³, —OR¹⁴, NR¹³R¹⁵, C₁-C₁₀-alkyl C₃-C₈-cycloalkyl, —NR¹⁶CX—R¹⁷, —NR¹⁸CX—OR¹⁹, —NR²⁰SOₘR²¹, —SOₚNR²²R²³ and —SO_{q}R²⁴,
m, p, q independently of one another denote 0, 1 or 2,
n denotes 0, 1, 2 or 3,
R³ denotes hydrogen or a group selected from among optionally substituted C₁-C₁₀-alkyl, C₆-C₁₀-aryl, heterocyclyl, C₃-C₈-cycloalkyl, —CX—C₁-C₁₀-alkyl and —CX—C₆-C₁₄-aryl,
R⁴, R⁵ independently of one another denote hydrogen, halogen or optionally substituted C₁-C₁₀-alkyl,
or
R⁴ and R⁵ together denote a C₃-C₈-alkyl bridge,
R⁶ denotes a group selected from among the general formulae

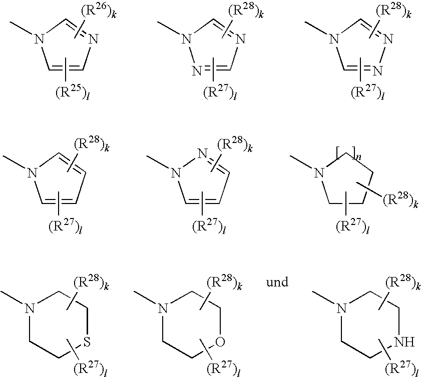

l,k independently of one another denote 1, 2 or 3,
R²⁵R²⁶R²⁷, R²⁸ independently of one another denote a group selected from among hydrogen, OH, halogen, CN and NO₂,
or
a group selected from among optionally substituted C₁-C₁₀-alkyl, C₆-C₁₈-aryl, heteroaryl, heterocyclyl, —CX—R¹⁷, —OR¹⁴, NR¹³R¹⁵, C₂-C₈-cycloalkyl, —NR²⁰SOₘR²¹, —SOₚNR²²R²³, SO_{q}R²⁴, —NR¹⁸CX—R¹⁹, —NR¹⁸CXOR¹⁷, while R²⁵ and R²⁶ cannot simultaneously represent hydrogen,
R⁸ denotes hydrogen or a group selected from among optionally substituted C₁-C₁₀-alkyl, C₆-C₁₈-aryl, —SO_{q}—C₁-C₁₀-alkyl, —SO_{q}—C₆-C₁₄-aryl, —CX—C₁-C₁₀-alkyl, —CX—C₆-C₁₄-aryl, C₆-C₁₀-aryl, heterocyclyl and C₃-C₈-cycloalkyl
R⁹ denotes hydrogen or a group selected from among optionally substituted C₁-C₁₀-alkyl, C₆-C₁₄-aryl, heteroaryl, C₃-C₈-cycloalkyl and heterocycloalkyl,
R¹² denotes hydrogen or a group selected from among optionally substituted benzyl, C₁-C₁₂-alkyl and C₆-C₁₄-aryl,
R⁷, R¹³, R¹⁵, R¹⁶, R¹⁸, R²⁰, R²², R²³ independently of one another denote hydrogen, or a group selected from among optionally substituted C₁-C₁₀-alkyl, C₆-C₁₄-aryl, heterocyclyl and C₃-C₈-cycloalkyl
R¹⁴, R¹⁹, R²⁹ independently of one another denote hydrogen or a group selected from among optionally substituted C₁-C₁₀-alkyl, C₆-C₁₄-aryl, C₃-C₈-cycloalkyl, heteroaryl, heterocyclyl, —CXNR₁₃R₁₅, —CXR₇
R¹⁷ denotes a group selected from among C₁-C₁₀-alkyl, C₆-C₁₄-aryl, heterocyclyl, heteroaryl and C₃-C₈-cycloalkyl
R²¹, R²⁴ independently denote hydrogen or OH, or a group selected from among optionally substituted N(C₁-C₁₀-alkyl)₂, N(C₃-C₈-cycloalkyl), C₁-C₁₀-alkyl, C₆-C₁₄-aryl, heterocyclyl, heteroaryl and C₃-C₈-cycloalkyl
and
X denotes O, S or NR²⁹
optionally in the form of the tautomers, the racemates, the enantiomers, the diastereomers and the mixtures thereof, as well as optionally the pharmacologically acceptable acid addition salts thereof.

Preferred are compounds of formula (Ia), wherein

R¹⁰, R¹¹ independently of one another denote hydrogen or halogen,
m, p, q denotes 0, 1 or 2
n denotes 0, 1, 2 or 3
R³ denotes hydrogen or C₁-C₅-alkyl
R⁴, R⁵ independently of one another denote hydrogen or C₁-C₅-alkyl,
R⁸ denotes a group selected from among hydrogen, C₁-C₅-alkyl, —SO_{q}—C₁-C₅-alkyl, —SO_{q}—C₆-C₁₄-aryl, phenyl and C₃-C₆-cycloalkyl
R⁹ denotes hydrogen or C₁-C₁₀-alkyl
R¹² denotes hydrogen or benzyl
R¹³, R¹⁵R¹⁶, R¹⁸ independently of one another denote a group selected from among hydrogen, C₁-C₅-alkyl, C₃-C₆-cycloalkyl and phenyl
R¹⁴, R¹⁹ independently of one another denote hydrogen or C₁-C₅-alkyl, and
R¹⁷ denotes optionally substituted C₁-C₅-alkyl or C₆-C₁₀-aryl.

Also preferred are compounds of formula (Ia), wherein

R¹⁰, R¹¹ denotes hydrogen
m, p, q denotes 0, 1 or 2
n denotes 0, 1, 2 or 3
R³ denotes hydrogen
R⁴, R⁵ independently of one another denote hydrogen or methyl,
R⁸ denotes hydrogen, —SO_{q}—C₆-C₁₄-aryl or —SO₂—C₁-C₅-alkyl
R⁹ denotes hydrogen
R¹² denotes hydrogen or benzyl,
R¹³, R¹⁵, R¹⁶, R¹⁸ independently of one another denote a group selected from among hydrogen, C₁-C₁₅-alkyl and phenyl,
R¹⁴, R¹⁹ independently of one another denote hydrogen or C₁-C₅-alkyl,
and
R¹⁷ denotes C₁-C₅-alkyl or C₆-C₁₄-aryl.

Particularly preferred are compounds of formula (Ia), wherein

R¹ denotes a group selected from among hydrogen, NO₂, NH₂, —NHCX—R¹⁷ and —NHSO₂R²¹.
R², denotes hydrogen or halogen
n denotes 2,
R³ denotes hydrogen
R⁴, R⁵ denote hydrogen or methyl
R⁶ denotes a group selected from among the general formulae

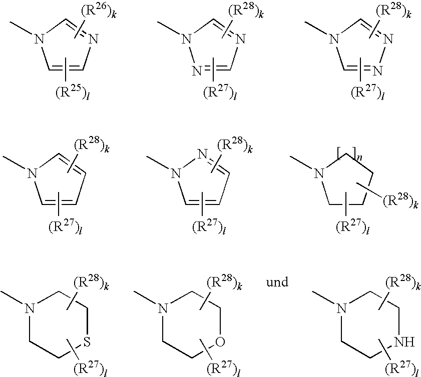

l,k denotes 1
R²⁶R²⁷ denotes hydrogen,
R⁸ denotes hydrogen or —SO₂CH₃,
R⁹ denotes hydrogen,
R¹⁰, R¹¹ denotes hydrogen, and
R¹² denotes hydrogen or benzyl

Also particularly preferred are compounds of formula (Ia), wherein

R⁶ denotes a group selected from among the general formulae

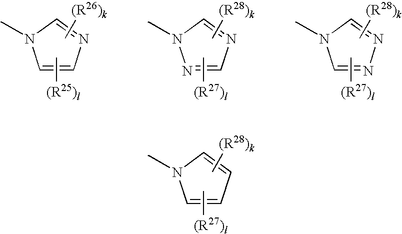

Particularly preferred are compounds of formula (Ia), wherein

R⁶ denotes an optionally substituted group of formula (j)

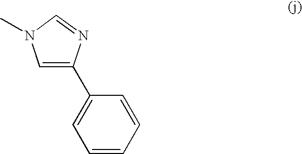

The term alkyl groups, including alkyl groups which are a part of other groups, denotes branched and unbranched alkyl groups with 1 to 10 carbon atoms, preferably 1-6, most preferably 1-4 carbon atoms, such as, for example: methyl, ethyl, propyl, butyl, pentyl, hexyl, heptyl, octyl, nonyl and decyl. Unless otherwise stated, the above-mentioned terms propyl, butyl, pentyl, hexyl, heptyl, octyl, nonyl and decyl include all the possible isomeric forms. For example, the term propyl includes the two isomeric groups n-propyl and iso-propyl, the term butyl includes n-butyl, iso-butyl, sec. butyl and tert.-butyl, the term pentyl includes iso-pentyl, neopentyl, etc.

In the above-mentioned alkyl groups one or more hydrogen atoms may optionally be replaced by other groups. For example, these alkyl groups may be substituted by the halogen atoms fluorine, chlorine, bromine or iodine. Preferably the substituents are fluorine or chlorine, most preferably chlorine. All the hydrogen atoms of the alkyl group may optionally also be replaced.

Similarly, in the above-mentioned alkyl groups, unless otherwise stated, one or more hydrogen atoms may optionally be replaced, for example, by an optionally substituted group selected from among OH, NO₂, CN, —O—C₁-C₅-alkyl, preferably —O-methyl or —O-ethyl, O—C₆-C₁₄-aryl, preferably O-phenyl, O-heteroaryl, preferably O-thienyl, O-thiazolyl, O-imidazolyl, O-pyridyl, O-pyrimidyl or O-pyrazinyl, saturated or unsaturated O-heterocycloalkyl, preferably O-pyrazolyl, O-pyrrolidinyl, O-piperidinyl, O-piperazinyl or O-tetrahydro-oxazinyl, C₆-C₁₄-aryl, preferably phenyl, heteroaryl, preferably thienyl, thiazolyl, imidazolyl, pyridyl, pyrimidyl or pyrazinyl, saturated or unsaturated heterocycloalkyl, preferably pyrazolyl, pyrrolidinyl, piperidinyl, piperazinyl or tetrahydro-oxazinyl, an amine group, preferably methylamine, benzylamine, phenylamine or heteroarylamine, saturated or unsaturated bicyclic ring systems, preferably benzimidazolyl and C₃-C₈-cycloalkyl, preferably cyclohexyl or cyclopropyl.

The term aryl denotes an aromatic ring system with 6 to 18 carbon atoms, preferably 6 to 14 carbon atoms, preferably 6 or 10 carbon atoms, most preferably phenyl, which, unless otherwise stated, may carry one or more of the following substituents, for example: OH, NO₂, CN, —OCHF₂, —OCF₃, —NH₂, halogen, for example fluorine, chlorine, bromine or iodine, preferably fluorine or chlorine, particularly preferably fluorine, C₁-C₁₀-alkyl, preferably C₁-C₅-alkyl, preferably C₁-C₃-alkyl, most preferably methyl or ethyl, —O—C₁-C₃-alkyl, preferably —O-methyl or —O-ethyl, —COOH or —CONH₂.

Examples of heteroaryl groups are 5-10-membered mono- or bicyclic heteroaryl rings wherein up to three C atoms may be replaced by one or more heteroatoms selected from among oxygen, nitrogen or sulphur, for example furan, thiophene, pyrrole, pyrazole, imidazole, triazole, tetrazole, pyridine, pyridazine, pyrimidine, pyrazine, triazine, oxazole, isoxazole, thiazole, thiadiazole, oxadiazole, while each of the above-mentioned heterocycles may optionally also be annellated to a benzene ring, preferably benzimidazole, and unless otherwise specified these heterocycles may for example carry one or more of the following substituents: OH, NO₂, CN, —NH₂, halogen, preferably fluorine or chlorine, C₁-C₁₀-alkyl, preferably C₁-C₅-alkyl, preferably C₁-C₃-alkyl, particularly preferably methyl or ethyl, —O—C₁-C₃-alkyl, preferably —O-methyl or —O-ethyl, —COOH, —COOCH₃, —CONH₂, —SO-alkyl, —SO₂-alkyl, —SO₂H, —SO₃-alkyl or optionally substituted phenyl.

Examples of cycloalkyl groups are saturated or unsaturated cycloalkyl groups with 3 to 8 carbon atoms, for example cyclopropyl, cyclobutyl, cyclopentyl, cyclopentenyl, cyclohexyl, cyclohexenyl, cycloheptyl or cyclooctyl, preferably cyclopropyl, cyclopentyl or cyclohexyl, while each of the above-mentioned cycloalkyl groups may optionally also carry one or more substituents or be annellated to a benzene ring.

Unless otherwise stated in the definitions, examples of heterocycloalkyl groups include 5-, 6- or 7-membered, saturated or unsaturated heterocycles which may contain nitrogen, oxygen or sulphur as heteroatoms, for example tetrahydrofuran, tetrahydrofuranone, γ-butyrolactone, α-pyran, γ-pyran, dioxolane, tetrahydropyran, dioxane, dihydrothiophene, thiolane, dithiolane, pyrroline, pyrrolidine, pyrazoline, pyrazolidine, imidazoline, imidazolidine, tetrazole, piperidine, pyridazine, pyrimidine, pyrazine, piperazine, triazine, tetrazine, morpholine, thiomorpholine, diazepan, oxazine, tetrahydro-oxazinyl, isothiazole and pyrazolidine, preferably pyrazolyl, pyrrolidinyl, piperidinyl, piperazinyl or tetrahydro-oxazinyl, while the heterocyclic group may optionally be substituted.

The halogen is generally fluorine, chlorine, bromine or iodine, preferably chlorine or fluorine, particularly preferably fluorine.

The compounds according to the invention may be present in the form of the individual optical isomers, mixtures of the individual enantiomers, diastereomers or racemates, in the form of the tautomers and also in the form of the free bases or the corresponding acid addition salts with pharmacologically acceptable acids—such as for example acid addition salts with hydrohalic acids, for example hydrochloric or hydrobromic acid, or organic acids, such as for example oxalic, fumaric, diglycolic, formic, malic, benzoic, benzenesulphonic, camphorsulphonic, acetic, ethanesulphonic, glutamic, maleic, mandelic, lactic, phosphoric, nitric, sulphuric, succinic, para-toluenesulphonic, trifluoroacetic, tartaric, citric or methanesulphonic acid.

The substituent R¹ may denote a group selected from among hydrogen, halogen, preferably fluorine or chlorine, CN, NO₂, and —NHCXNH₂, preferably NHCONH₂ or a group selected from among optionally substituted —COR⁷, —COOR⁷, —CONR⁷R¹³, —OR¹⁴, preferably OH, NR¹³R¹⁵, C₁-C₁₀-alkyl, C₃-C₈-cycloalkyl, —NR¹⁶CX—R¹⁷, —NR¹⁸CX—OR¹⁹, —NR²⁰SOₘR²¹, —SOₚNR²²R²³ preferably —SO₂NHR²³, and —SO_{q}R², particularly preferably the substituent R¹ denotes —NR²⁰SOₘR²¹, preferably —NHSOₘR²¹.

The substituent R² may denote a group selected from among hydrogen, halogen, preferably fluorine or chlorine, CN, NO₂, and —NHCXNH₂, preferably NHCONH₂ or a group selected from among optionally substituted —COR⁷, —COOR⁷, —CONR⁷R¹³, —OR¹⁴, preferably OH, NR¹³R¹⁵, C₁-C₁₀-alkyl, C₃-C₈-cycloalkyl, —NR¹⁶CX—R¹⁷, —NR¹⁸CX—OR¹⁹, —NR²⁰SOₘR²¹, —SOₚNR²²R²³, preferably —SO₂NHR²³ and —SO_{q}R²³. Particularly preferably the substituent R² denotes hydrogen or fluorine.

The substituents R¹⁰ and R¹¹ may be identical or different and denote a group selected from among hydrogen, halogen, preferably fluorine or chlorine, CN, NO₂, and —NHCXNH₂, preferably NHCONH₂ or a group selected from among optionally substituted —COR⁷, —COOR⁷, —CONR⁷R¹³, —OR¹⁴, preferably OH, NR¹³R¹⁵, C₁-C₁₀-alkyl, C₃-C₈-cycloalkyl, —NR¹⁶CX—R¹⁷, —NR¹⁸CX—OR¹⁹, —NR²⁰SOₘR²¹, —SOₚNR²²R²³ preferably —SO₂NHR²³ and —SO_{q}R². Particularly preferably the substituents R¹⁰ and R¹¹ denote hydrogen.

The variable m, p and q may represent 0, 1 or 2, preferably 2.

The variable n may represent 0, 1, 2 or 3, preferably 2.

The substituent R³ may represent hydrogen or a group selected from among optionally substituted C₁-C₁₀-alkyl, C₆-C₁₀-aryl, heterocyclyl and C₃-C₈-cycloalkyl, —CX—C₁-C₁₀-alkyl, —CX—C₆-C₁₄-aryl.

Preferably the substituent R³ denotes hydrogen.

The substituents R⁴ and R⁵ may be identical or different and denote hydrogen, halogen or optionally substituted C₁-C₁₀-alkyl, preferably hydrogen or C₁-C₁₀-alkyl, particularly preferably hydrogen or methyl,

or
R⁴ and R⁵ may together form a C₃-C₈-alkyl bridge, preferably a cyclohexyl, cyclopentyl or cyclopropyl bridge.

The substituent R⁶ may represent a group selected from among the general formulae

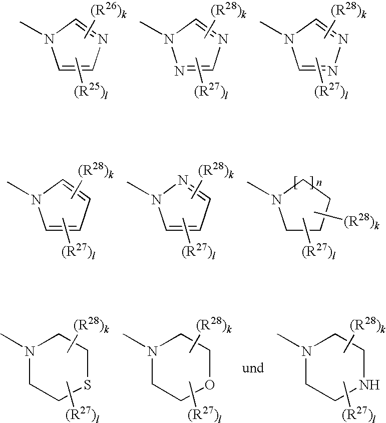

wherein
the variables l and k independently of one another denote 1, 2 or 3, preferably 1.
R⁶ particularly preferably denotes

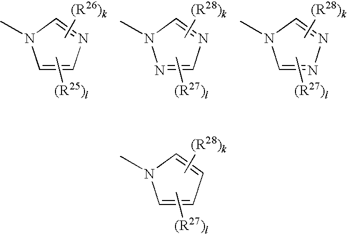

R⁶ especially preferably denotes

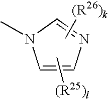

The substituents R²⁵R²⁶R²⁷, R²⁸ may be identical or different and denote a group selected from among hydrogen, OH, halogen, CN and NO₂,

or
a group selected from among optionally substituted C₁-C₁₀-alkyl, C₆-C₁₈-aryl, preferably phenyl, heteroaryl, preferably pyridyl, heterocyclyl, —CX—R¹⁷, —OR¹⁴, NR¹³R¹⁵, C₂-C₈-cycloalkyl, —NR²⁰SOₘR²¹, SOₚNR²²R²³—SO_{q}R²⁴, —NR¹⁸CX—R¹⁹, —NR¹⁸CXOR¹⁷, while R²⁵ and R²⁶ cannot simultaneously represent hydrogen.

The substituent R⁸ may represent hydrogen or a group selected from among optionally substituted C₁-C₁₀-alkyl, C₆-C₁₈-aryl, —SO_{q}—C₁-C₁₀-alkyl, —SO_{q}—C₆-C₁₄-aryl, —CX—C₁-C₁₀-alkyl, —CX—C₆-C₁₄-aryl, C₆-C₁₀-aryl, heterocyclyl and C₃-C₈-cycloalkyl, preferably hydrogen or —SO₂CH₃.

The substituent R⁹ may represent hydrogen or a group selected from among optionally substituted C₁-C₁₀-alkyl, C₆-C₁₄-aryl, heteroaryl, C₃-C₈-cycloalkyl and heterocycloalkyl, preferably hydrogen.

The substituent R¹² may represent hydrogen or a group selected from among optionally substituted benzyl, C₁-C₁₂-alkyl and C₆-C₁₄-aryl, CX—C₁-C₁₂-alkyl and CX—C₆-C₁₄-aryl, preferably hydrogen.

The substituents R⁷, R¹³, R¹⁵R¹⁶, R¹⁸, R²⁰, R²², R²³ and R²⁴ may be identical or different and represent hydrogen, or

a group selected from among optionally substituted C₁-C₁₀-alkyl, C₆-C₁₄-aryl, heterocyclyl and C₃-C₈-cycloalkyl.

Particularly preferably the substituent R²⁰ denotes methyl, ethyl or isopropyl.

The substituents R¹⁴, R¹⁹ and R²⁹ may be identical or different and represent hydrogen or a group selected from among optionally substituted C₁-C₁₀-alkyl, preferably methyl or difluoromethyl, C₆-C₁₄-aryl, C₃-C₈-cycloalkyl, heteroaryl, heterocyclyl, —CXNR₁₃R₁₅,

particularly preferably the substituent R¹⁴ denotes methyl or difluoromethyl.

The substituent R¹⁷ may represent a group selected from among C₁-C₁₀-alkyl, preferably methyl or ethyl, C₆-C₁₄-aryl, heterocyclyl, heteroaryl and C₃-C₈-cycloalkyl.

The substituent R²¹ may represent hydrogen or OH, or

a group selected from among optionally substituted N(C₁-C₁₀-alkyl)₂, N(C₃-C₈-cycloalkyl), C₁-C₁₀-alkyl, C₆-C₁₄-aryl, heterocyclyl, heteroaryl and C₃-C₈-cycloalkyl.
X may represent O, S or NR²⁹, preferably O.

The preparation of the compounds according to the invention of formula (Ia) may be found in WO 2004/039784.

Examples of compounds of formula (Ia) are listed in the following Tables 1, 2 and 3. The abbreviations X₁, X₂, X₄, X₅, X₆, X₈ and X₁₂ used in the Tables each represent a linkage to a position in the general formula given under Table 1 instead of the corresponding groups R¹, R², R⁴, R⁵, R⁶, R⁸ and R¹².

TABLE 1

| | |
|---|---|
| | (IA) |
| 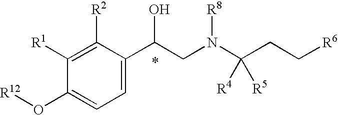 | |

| | | | | | | | | Stereo- |
|---|---|---|---|---|---|---|---|---|
| Ex. | R1 | R2 | R4 | R5 | R6 | R8 | R12 | chem* |
| 1 | 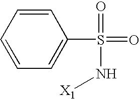 | H | 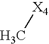 | 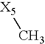 | 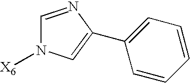 | H | H | R |
| 2 | 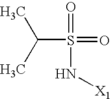 | H | 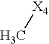 | 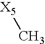 | 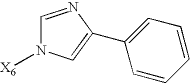 | H | H | R |
| 3 | 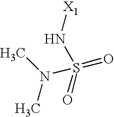 | H | 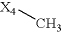 | 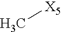 | 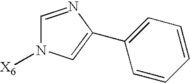 | H | H | R |
| 4 | 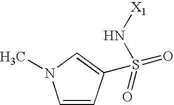 | H | 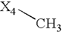 | 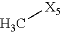 | 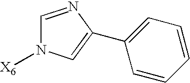 | H | H | R |
| 5 | 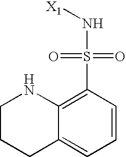 | H | 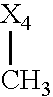 | 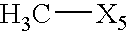 | 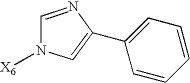 | H | H | R |
| 6 | 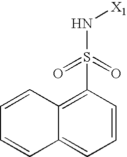 | H | 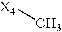 | 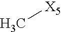 | 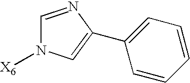 | H | H | R |
| 7 | 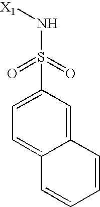 | H | 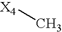 | 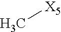 | 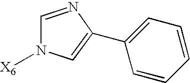 | H | H | R |
| 8 | 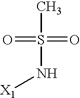 | H | H | H | 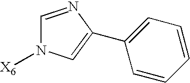 | 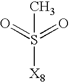 | H | R |
| 9 | 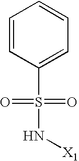 | H | H | H | 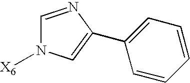 | H | H | R |
| 10 | 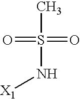 | H | H | H | 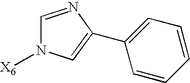 | H | H | R |
| 11 | 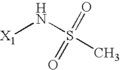 | H | H | 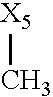 | 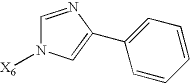 | H | H | R |
| 12 | 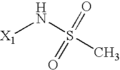 | H | 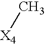 | 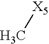 | 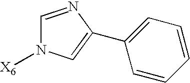 | H | H | rac |
| 13 | 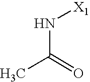 | H | 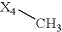 | 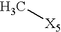 | 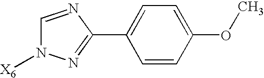 | H | H | R |
| 14 | 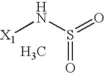 | H | 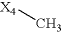 | 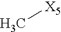 | 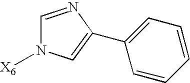 | H | 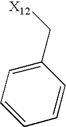 | R |
| 15 | 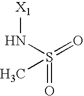 | H | 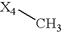 | 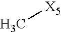 | 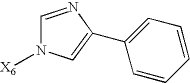 | H | H | R |
| 16 | 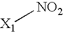 | H | 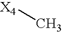 | 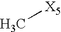 | 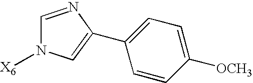 | H | 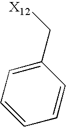 | R |
| 17 | 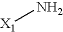 | H | 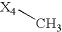 | 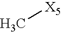 | 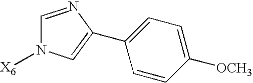 | H | 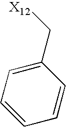 | R |
| 18 | 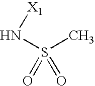 | H | 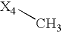 | 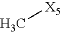 | 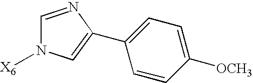 | H | 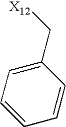 | R |
| 19 | 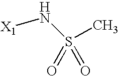 | H | 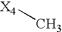 | 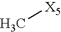 | 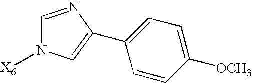 | H | H | R |
| 20 | 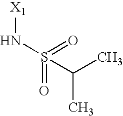 | H | 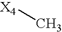 | 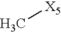 | 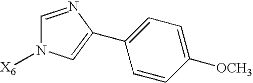 | H | 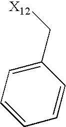 | S |
| 21 | 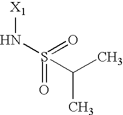 | H | 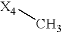 | 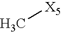 | 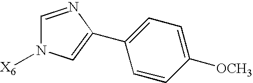 | H | H | S |
| 22 | 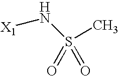 | H | 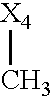 | 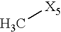 | 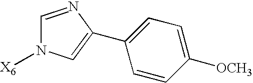 | H | H | S |
| 23 | 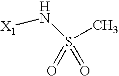 | H | 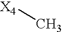 | 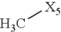 | 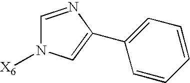 | H | H | S |
| 24 | 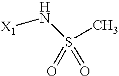 | H | 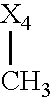 | 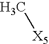 | 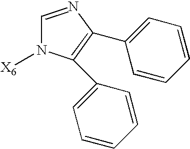 | H | H | rac |
| 25 | H | F | 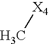 | 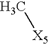 | 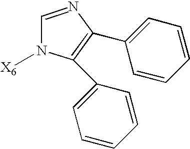 | H | H | rac |
| 26 | H | F | 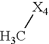 | 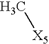 | 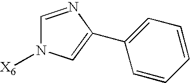 | H | H | rac |
| 27 | 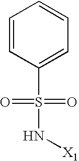 | H | 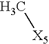 | 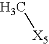 | 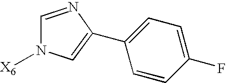 | H | 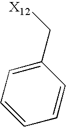 | rac |
| 28 | 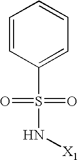 | H | 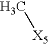 | 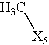 | 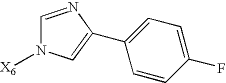 | H | H | rac |

TABLE 2

| | |
|---|---|
| | (IB) |
| 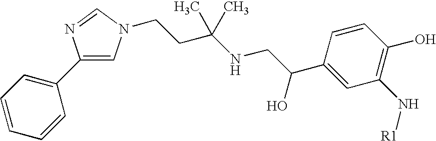 | |

| | | Mass spec. |
|---|---|---|
| Example | R1 | determined M.W. |
| 29 | 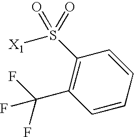 | 589 |
| 30 | 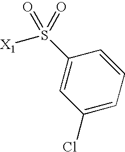 | 555 |
| 31 | 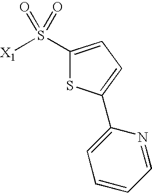 | 604 |
| 32 | 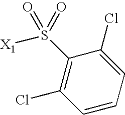 | 589 |
| 33 | 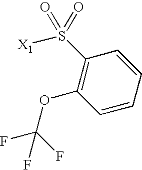 | 605 |
| 34 | 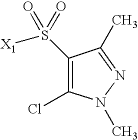 | 573 |
| 35 | 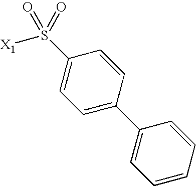 | 597 |
| 36 | 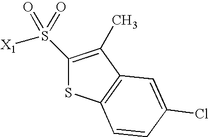 | 625 |
| 37 | 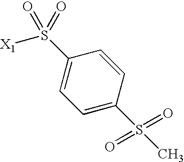 | 599 |
| 38 | 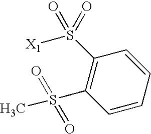 | 599 |
| 39 | 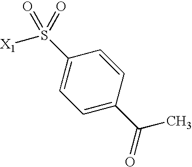 | 563 |
| 40 | 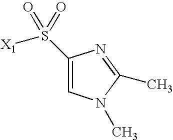 | 539 |
| 41 | 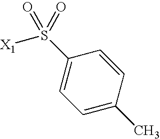 | 535 |
| 42 | 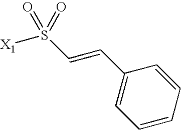 | 547 |
| 43 | 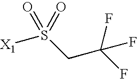 | 525 |
| 44 | 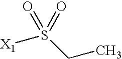 | 473 |
| 45 | 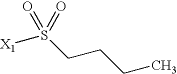 | 501 |
| 46 | 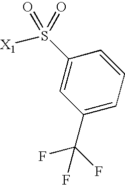 | 589 |
| 47 | 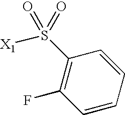 | 539 |
| 48 | 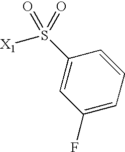 | 539 |
| 49 | 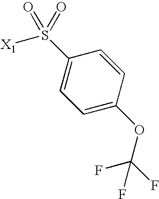 | 605 |
| 50 | 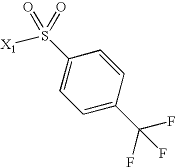 | 589 |
| 51 | 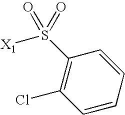 | 555 |
| 52 | 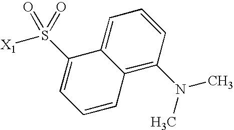 | 614 |
| 53 | 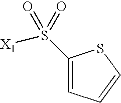 | 527 |
| 54 | 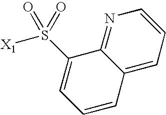 | 662 |
| 55 | 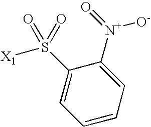 | 566 |
| 56 | 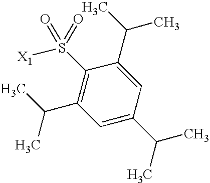 | 647 |
| 57 | 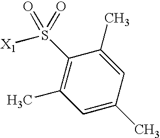 | 563 |
| 58 | 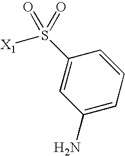 | 536 |
| 59 | 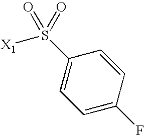 | 539 |
| 60 | 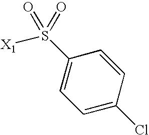 | 555 |
| 61 | 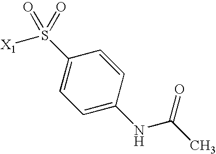 | 578 |
| 62 | 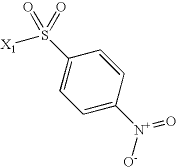 | 566 |
| 63 | 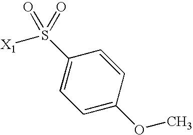 | 551 |
| 64 | 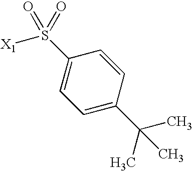 | 577 |
| 65 | 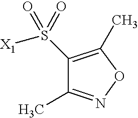 | 539 |
| 66 | 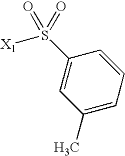 | 535 |
| 67 | 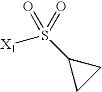 | 485 |
| 68 | 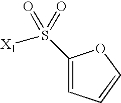 | 601 |
| 69 | 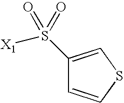 | 527 |
| 70 | 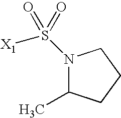 | 528 |
| 71 | 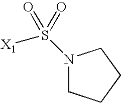 | 514 |
| 72 | 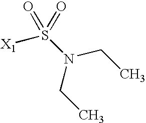 | 516 |
| 73 | 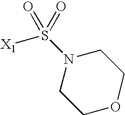 | 528 |
| 74 | 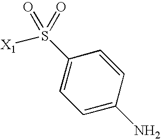 | 536 |
| 75 | 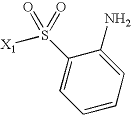 | 534 |

TABLE 3

| | |
|---|---|
| | (IC) |
| 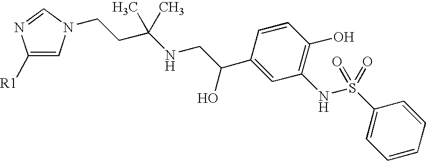 | |

| | | Mass spec. |
|---|---|---|
| Example | R1 | determined M.W. |
| 76 | 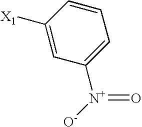 | 566 |
| 77 | 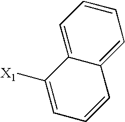 | 571 |
| 78 | 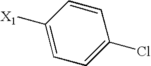 | 555 |
| 79 | 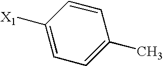 | 535 |
| 80 | 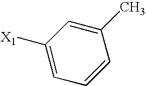 | 535 |
| 81 | 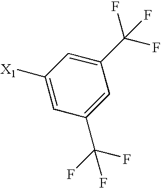 | 657 |
| 82 | 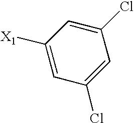 | 589 |
| 83 | 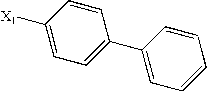 | 597 |
| 84 | 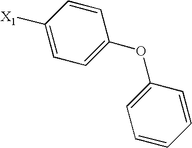 | 613 |
| 85 | 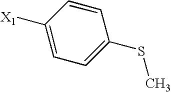 | 567 |
| 86 | 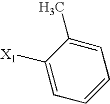 | 535 |
| 87 | 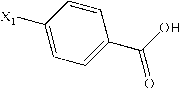 | 565 |
| 88 | 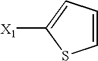 | 527 |
| 89 | 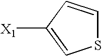 | 527 |
| 90 | 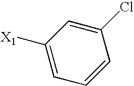 | 555 |
| 91 | 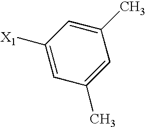 | 549 |
| 92 | 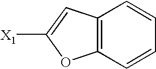 | 561 |
| 93 | 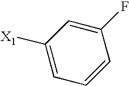 | 539 |
| 94 | 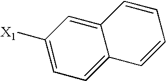 | 571 |
| 95 | 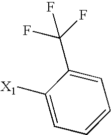 | 589 |
| 96 | 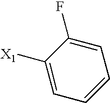 | 539 |
| 97 | 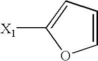 | 511 |
| 98 | 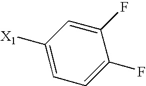 | 557 |
| 99 | 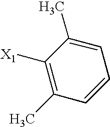 | 549 |
| 100 | 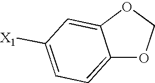 | 565 |
| 101 | 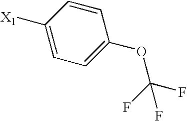 | 605 |
| 102 | 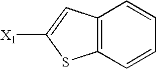 | 577 |
| 103 | 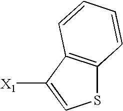 | 577 |
| 104 | 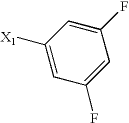 | 557 |
| 105 | 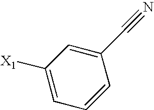 | 546 |
| 106 | 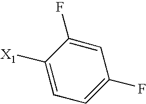 | 557 |
| 107 | 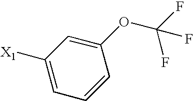 | 605 |
| 108 | 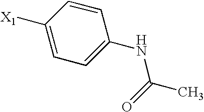 | 578 |
| 109 | 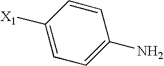 | 536 |

The present invention further relates to the use of compounds of general formula (Ib)

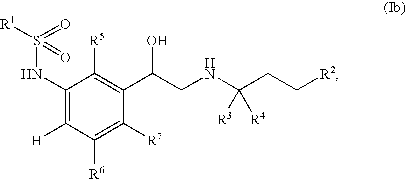

wherein
R¹ denotes an optionally substituted aryl or heteroaryl group,
R² an optionally substituted heteroaryl or heterocyclyl group, wherein R² contains at least one nitrogen atom,
R³ and R⁴ independently of one another denote a hydrogen atom or an optionally substituted group selected from among C₁-C₅-alkyl, C₃-C₆-cycloalkyl, heterocyclyl, aryl and heteroaryl or
R³ and R⁴ together denote a 2- to 7-membered alkylene bridge,
R⁵, R⁶ and R⁷ independently of one another denote a hydrogen atom or a group selected from among optionally substituted C₁-C₁₀-alkyl, alkenyl, alkynyl, C₆-C₁₀-aryl, heterocyclyl, C₃-C₈-cycloalkyl, —NR⁸—(C₁-C₅-alkyl), —NR³-aryl, halogen, cyano, —NR⁸CO—(C₁-C₅-alkyl), —NR⁸CO-aryl, —NR⁸SO₂—(C₁-C₅-alkyl), —NR⁸SO₂-aryl, —CO₂R⁸, —SO₂R⁸, —CONHR⁸, —SO₂NHR⁸ and —OR⁸, wherein the above-mentioned alkyl groups may each be substituted, and
R⁸ denotes a hydrogen atom or a C₁-C₅-alkyl group,
optionally in the form of the tautomers, the racemates, the enantiomers, the diastereomers, the solvates and hydrates thereof and the mixtures thereof, as well as optionally the prodrugs, double prodrugs and salts thereof, particularly the physiologically acceptable salts thereof with inorganic or organic acids or bases.

Preferred are compounds of general formula (Ib), wherein

R² to R⁷ are as hereinbefore defined and
R¹ denotes an optionally substituted phenyl group.

Another preferred sub-group comprises the compounds of general formula (Ib), wherein

R¹ and R³ to R⁷ are as hereinbefore defined,
R² denotes a group selected from among the optionally substituted groups of formula

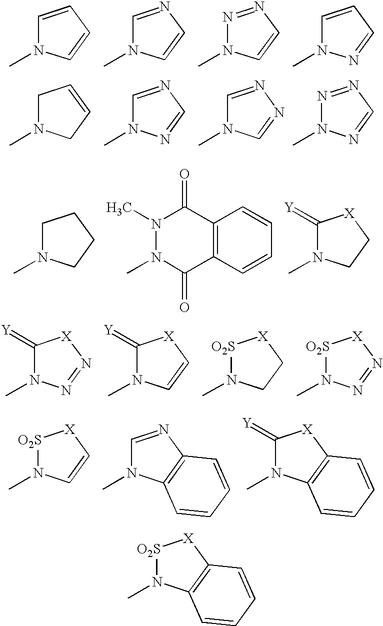

wherein the above-mentioned groups may each be substituted by one or more groups R¹⁰ and
R¹⁰ denotes OH, NO₂, CN, —OCHF₂, —OCF₃, —NH₂, —NH-alkyl, —N(-alkyl)-alkyl, —NH-aryl, —N(-alkyl)-aryl, —NHCO-alkyl, —NHCO₂-alkyl, —NHCO-aryl, —N(-alkyl)-CO-alkyl, —N(-alkyl)-CO-aryl, —NHSO₂-alkyl, —NHSO₂-aryl, —N(-alkyl)-SO₂-alkyl, —N(-alkyl)-SO₂-aryl, —CO₂-alkyl, —SO₂-alkyl, —SO₂-aryl, —CONH-alkyl, —CONH-aryl, —CON(-alkyl)-alkyl, —CON(-alkyl)-aryl, —SO₂NH-alkyl, —SO₂NH-aryl, —SO₂N(-alkyl)-alkyl, —SO₂N(-alkyl)-aryl, —O-alkyl, —O-aryl, —S-alkyl, —S-aryl, halogen, C₁-C₁₀-alkyl, —O—(C₁-C₃-alkyl), —COOH, —CONH₂, —CON(-alkyl)-SO₂-alkyl, —CONHSO₂-alkyl, —CONHOH, 2,5-dihydro-5-oxo-4H-1,2,4-oxadiazol-3-yl, 2,5-dihydro-5-oxo-4H-1,2,4-thiadiazol-3-yl, 2,5-dihydro-2-oxo-3H-1,2,4,5-oxathiadiazol-4-yl, 1-acetyl-2-amino-propen-1-yl, tetrazolyl, heterocyclyl, aryl or heteroaryl,
and wherein X denotes an oxygen atom or an —NR⁹ group and
Y denotes an oxygen or sulphur atom,
   and R⁹ denotes a hydrogen atom or a group selected from among C₁-C₁₀-alkyl, C₃-C₈-cycloalkyl, heterocyclyl, aryl or heteroaryl, while the groups mentioned for R⁹ hereinbefore may each be substituted by one of the groups mentioned for R¹⁰.

Particularly preferred are compounds of general formula (Ib), wherein

R¹ and R² as well as R⁵ to R⁷ are as hereinbefore defined, and
R³ and R⁴ independently of one another denote a hydrogen atom or a methyl or ethyl group or
R³ and R⁴ together denote a 2- to 5-membered alkylene bridge.

Particularly preferred are compounds of general formula (Ib), wherein

R¹ to R⁴ are as hereinbefore defined, and
R⁵, R⁶ and R⁷ independently of one another denote hydrogen, optionally substituted C₁-C₁₀-alkyl, halogen, CN, —NR⁸CO—(C₁-C₅-alkyl), —NR⁸SO₂—(C₁-C₅-alkyl), —CO₂R⁸, —SO₂R⁸, —CONHR⁸, —SO₂NHR⁸ or —OR⁸ and
R⁸ denotes a hydrogen atom or a C₁-C₅-alkyl group represent.

Also preferred are compounds of general formula (Ib), wherein

R¹ denotes a phenyl group optionally substituted by a halogen atom or a cyano or nitro group,
R² denotes a group selected from among the optionally substituted groups of formula n:

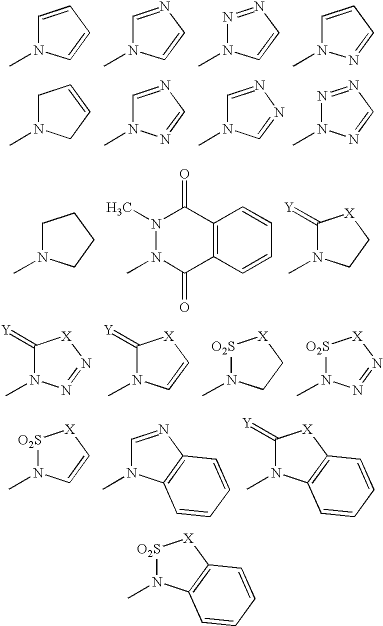

wherein the above-mentioned groups may each be substituted by one or more groups R¹⁰ and
R¹⁰ denotes OH, NO₂, CN, —OCHF₂, —OCF₃, —NH₂, —NH-alkyl, —N(-alkyl)-alkyl, —NH-aryl, —N(-alkyl)-aryl, —NHCO-alkyl, —NHCO₂-alkyl, —NHCO-aryl, —N(-alkyl)CO-alkyl, —N(-alkyl)-CO-aryl, —NHSO₂-alkyl, —NHSO₂-aryl, —N(-alkyl)-SO₂-alkyl, —N(-alkyl)-SO₂-aryl, —CO₂-alkyl, —SO₂-alkyl, —SO₂-aryl, —CONH-alkyl, —CONH-aryl, —CON(alkyl)-alkyl, —CON(-alkyl)-aryl, —SO₂NH-alkyl, —SO₂NH-aryl, —SO₂N(-alkyl)-alkyl, —SO₂N(-alkyl)-aryl, —O-aryl, —S-alkyl, —S-aryl, halogen, C₁-C₁₀-alkyl, —O—(C₁-C₃-alkyl), —COOH, —CONH₂, —CON(-alkyl)-SO₂-alkyl, —CONHSO₂-alkyl, —CONHOH, 2,5-dihydro-5-oxo-4H-1,2,4-oxadiazol-3-yl, 2,5-dihydro-5-oxo-4H-1,2,4-thiadiazol-3-yl, 2,5-dihydro-2-oxo-3H-1,2,4,5-oxathia-diazol-4-yl, 1-acetyl-2-amino-propen-1-yl, tetrazolyl, heterocyclyl, aryl or heteroaryl,
and wherein X denotes an oxygen atom or an —NR⁹ group and
Y denotes an oxygen or sulphur atom,
R³ and R⁴ independently of one another each denote a methyl or ethyl group or
R³ and R⁴ together denote an ethylene bridge,
R⁵, R⁶ and R⁷ independently of one another each denote a hydrogen, fluorine or chlorine atom or a cyano, methoxy, methanesulphonylamino, methanesulphonyl, difluoromethoxy, trifluoromethoxy, difluoromethyl or trifluoromethyl group and
R⁹ denotes a hydrogen atom or an optionally substituted aryl or optionally substituted heteroaryl group.

Particularly preferred are compounds of general formula (Ib), wherein

R¹ denotes a phenyl group optionally substituted by a fluorine, chlorine, bromine or iodine atom or a cyano or nitro group,
R² denotes a group selected from among the groups of formula (I)-(vi):

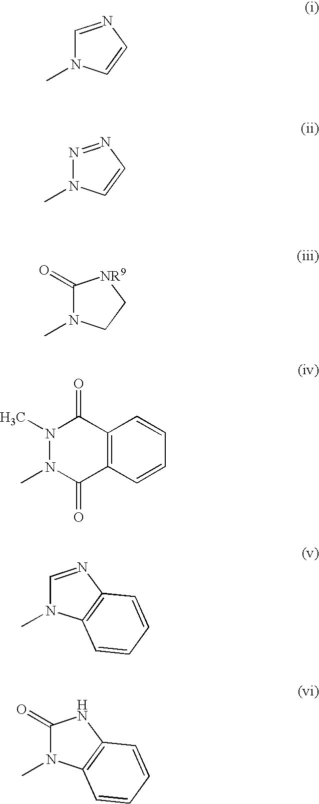

wherein R⁹ denotes a phenyl or pyridyl group optionally substituted by a fluorine atom or an amino, nitro, hydroxy or methoxy group and wherein the above-mentioned groups (i) to (vi) may be substituted in each case by one or two groups R¹⁰ and
R¹⁰ denotes OH, NO₂, CN, —OCHF₂, —OCF₃, —NH₂, —NH-alkyl, —N(alkyl)-alkyl, —NH-aryl, —N(alkyl)-aryl, —NHCO-alkyl, —NHCO₂-alkyl, —NHCO-aryl, —N(-alkyl)-CO-alkyl, —N(-alkyl)-CO-aryl, —NHSO₂-alkyl, —NHSO₂-aryl, —N(-alkyl)-SO₂-alkyl, —N(-alkyl)-SO₂-aryl, —CO₂-alkyl, —SO₂-alkyl, —SO₂-aryl, —CONH-alkyl, —CONH-aryl, —CON(alkyl)-alkyl, —CON(-alkyl)-aryl, —SO₂NH-alkyl, —SO₂NH-aryl, —SO₂N(-alkyl)-alkyl, —SO₂N(-alkyl)-aryl, —O-aryl, —S-alkyl, —S-aryl, halogen, C₁-C₁₀-alkyl, —O—(C₁-C₃-alkyl), —COOH, —CONH₂, —CON(-alkyl)-SO₂-alkyl, —CONHSO₂-alkyl, —CONHOH, 2,5-dihydro-5-oxo-4H-1,2,4-oxadiazol-3-yl, 2,5-dihydro-5-oxo-4H-1,2,4-thiadiazol-3-yl, 2,5-dihydro-2-oxo-3H-1,2,4,5-oxathiadiazol-4-yl, 1-acetyl-2-amino-propen-1-yl, tetrazolyl, heterocyclyl, aryl or heteroaryl,
R³ and R⁴ independently of one another denote a methyl or ethyl group or
R³ and R⁴ together denote an ethylene bridge and
R⁵, R⁶ and R⁷ independently of one another represent a hydrogen, fluorine or chlorine atom or a cyano, methoxy, methanesulphonylamino, methanesulphonyl, difluoromethoxy, trifluoromethoxy, difluoromethyl or trifluoromethyl group.

Particularly preferred are compounds of general formula (Ib), wherein

R¹ denotes a phenyl group optionally substituted by a fluorine, chlorine, bromine or iodine atom or a cyano or nitro group,
R² denotes a group selected from among the groups of formula n (i)-(vi):

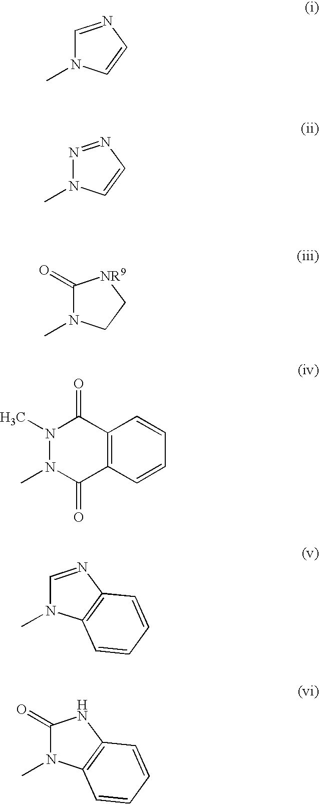

wherein R⁹ denotes a phenyl or pyridyl group optionally substituted by a fluorine atom or an amino, nitro, hydroxy or methoxy group, and wherein the above-mentioned groups (i) to (vi) may each be substituted by one or two groups R¹⁰ and
R¹⁰ denotes OH, —NO₂, —CN, —NH₂, —I, —N(CH₃)₂, —NHCO₂CH₃, —NHSO₂CH₃, C₁-C₃-alkyl, —SO₂N(CH₃)₂, —CO₂H, benzyloxycarbonyl, ethoxycarbonyl, methoxycarbonyl, —CONHOH, tetrazol-5-yl, pyridinyl, methoxy-pyridinyl, phenyl optionally substituted by hydroxy, fluorine, methoxy, amino, nitro, dimethylamino, methylcarbonylamino, methylsulphonylamino, dimethylamino-sulphonylamino, carboxy, ethoxycarbonyl, benzyloxycarbonyl, hydroxy-aminocarbonyl or tetrazol-5-yl, or thiophenyl, 5-methyl-thiophen-2-yl, 3,5-dimethyl-isoxazol-4-yl or 1-acetyl-2-amino-propenyl,
   R³ and R⁴ each denote a methyl or ethyl group or
   R³ and R⁴ together denote an ethylene bridge and
   R⁵, R⁶ and R⁷ each represent a hydrogen atom.

Mention should be made most particularly of compounds of general formula (Ib), wherein

R¹ denotes a phenyl group,
R² denotes a group selected from among the groups of formula n (i)-(iii) or (v):

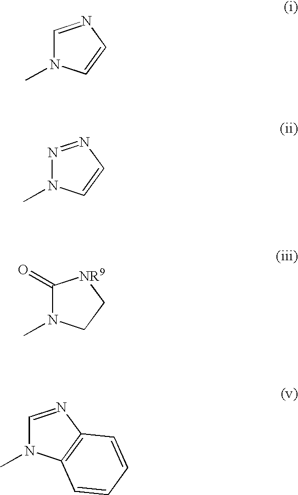

wherein R⁹ denotes a phenyl or pyridyl group optionally substituted by a fluorine atom or an amino, nitro, hydroxy or methoxy group,
and wherein the above-mentioned groups (i) to (iii) and (v) may each be substituted by a group R¹⁰ and
R¹⁰ denotes an iodine atom or a nitro, amino, methyl, carboxy, methoxycarbonyl, ethoxycarbonyl, pyridin-4-yl, pyridin-2-yl, 6-methoxy-pyridin-3-yl, thiophen-2-yl, 5-methyl-thiophen-2-yl, 3.5-dimethyl-isoxazol-4-yl, 1-acetyl-2-amino-propen-1-yl or a phenyl group, while the phenyl group may be substituted, preferably in the 4 position, by a fluorine atom or by a hydroxy, methoxy, nitro, amino, dimethylamino, methylcarbonylamino, methylsulphonylamino, dimethylamino-sulphonylamino, carboxy, ethoxycarbonyl, benzyloxycarbonyl, hydroxyaminocarbonyl or tetrazol-5-yl group,
   R³ and R⁴ each denote a methyl group and
   R⁵, R⁶ and R⁷ each represent a hydrogen atom,
   but particularly those compounds of general formula (Ib) wherein
   R¹ denotes a phenyl group,
   R² denotes a group of formulae (Ia) or (v):

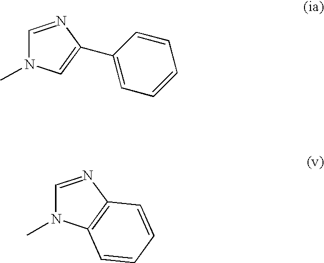

wherein the above-mentioned group (i) in the phenyl moiety may be substituted by a fluorine atom or by a hydroxy, methoxy, nitro, amino, dimethylamino, methylcarbonylamino, methylsulphonylamino, dimethylaminosulphonylamino, carboxy, ethoxycarbonyl, benzyloxycarbonyl, hydroxy-aminocarbonyl or tetrazol-5-yl group and
the above-mentioned group (v) may be substituted in the benzyl moiety by a nitro, amino, carboxy or C₁₋₂-alkyloxy-carbonyl group,
   R³ and R⁴ each denote a methyl group and
   R⁵, R⁶ and R⁷ each represent a hydrogen atom.

Particularly preferred are the following compounds of formula (Ib) according to Table 4:

The abbreviation X₂ used in Table 4 denotes a linkage to the position in the general formula given under Table 4 instead of the corresponding group R².

TABLE 4

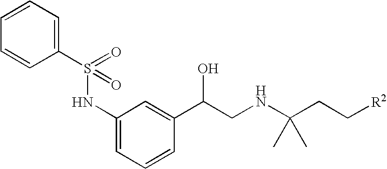

| Example | R² |
|---|---|
| 1 | 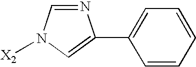 |
| 2 | 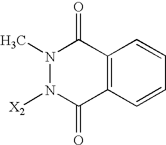 |
| 3 | 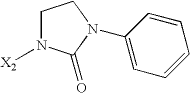 |
| 4 | 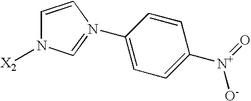 |
| 5 | 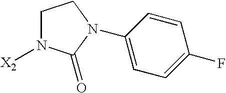 |
| 6 | 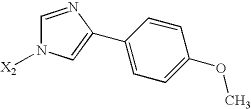 |
| 7 | 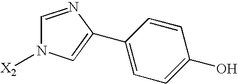 |
| 8 | 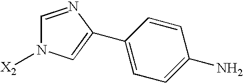 |
| 9 | 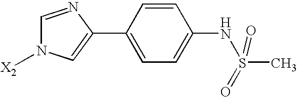 |
| 10 | 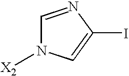 |
| 11 | 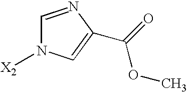 |
| 12 | 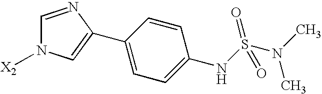 |
| 13 | 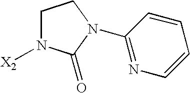 |
| 14 | 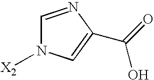 |
| 15 | 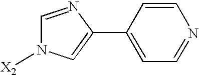 |
| 16 | 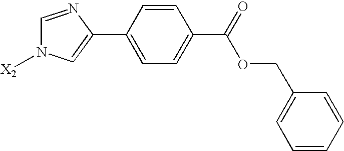 |
| 17 | 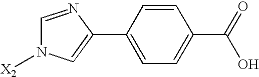 |
| 18 | 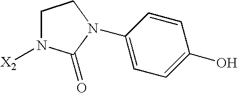 |
| 19 | 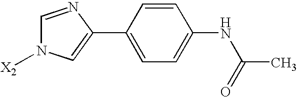 |
| 20 | 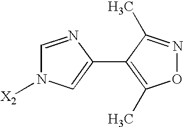 |
| 21 | 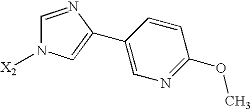 |
| 22 | 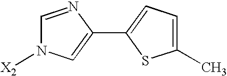 |
| 23 | 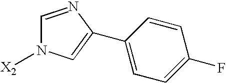 |
| 24 | 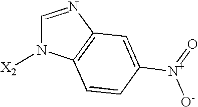 |
| 25 | 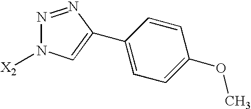 |
| 26 | 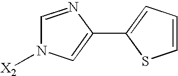 |
| 27 | 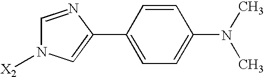 |
| 28 | 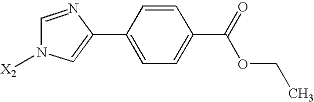 |
| 29 | 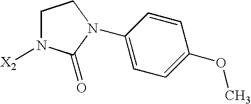 |
| 30 | 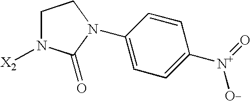 |
| 31 | 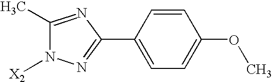 |
| 32 | 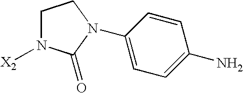 |
| 33 | 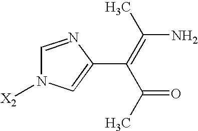 |
| 34 | 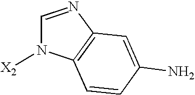 |
| 35 | 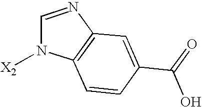 |
| 36 | 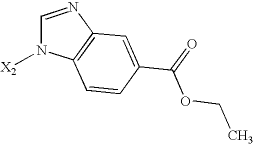 |
| 37 | 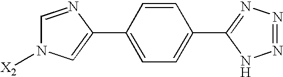 |
| 38 | 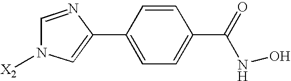 |

N-(3-{-[1,1-dimethyl-3-(4-phenyl-imidazol-1-yl)-propylamino]-1-hydroxy-ethyl}-phenyl)-benzenesulphonamide
N-(3-{2-[1,1-dimethyl-3-(3-methyl-1,4-dioxo-3,4-dihydro-1H-phthalazin-2-yl)-propylamino]-1-hydroxy-ethyl}-phenyl)-benzenesulphonamide
N-(3-{2-[1,1-dimethyl-3-(2-oxo-3-phenyl-imidazolidin-1-yl)-propylamino]-1-hydroxy-ethyl}-phenyl)-benzenesulphonamide
N-[3-(2-{1,1-dimethyl-3-[4-(4-nitro-phenyl)-imidazol-1-yl]-propylamino}-1-hydroxy-ethyl)-phenyl]-benzenesulphonamide
N-[3-(2-{3-[3-(4-fluoro-phenyl)-2-oxo-imidazolidin-1-yl]-1,1-dimethyl-propylamino}-1-hydroxy-ethyl)-phenyl]-benzenesulphonamide
N-[3-(1-hydroxy-2-{3-[4-(4-methoxy-phenyl)-imidazol-1-yl]-1,1-dimethyl-propylamino}ethyl)-phenyl]-benzenesulphonamide
N-[3-(1-hydroxy-2-{3-[4-(4-hydroxy-phenyl)-imidazol-1-yl]-1,1-dimethyl-propylamino}-ethyl)-phenyl]-benzenesulphonamide
N-[3-(2-{3-[4-(4-amino-phenyl)-imidazol-1-yl]-1,1-dimethyl-propylamino}-1-hydroxy-ethyl)-phenyl]-benzenesulphonamide
N-[3-(1-hydroxy-2-{3-[4-(4-methanesulphonylamino-phenyl)-imidazol-1-yl]-1,1-dimethyl-propylamino}-ethyl)-phenyl]-benzenesulphonamide
N-(3-{1-hydroxy-2-[3-(4-iodo-imidazol-1-yl)-1,1-dimethyl-propylamino]-ethyl}-phenyl)-benzenesulphonamide
methyl 1-{3-[2-(3-benzenesulphonylamino-phenyl)-2-hydroxy-ethylamino]-3-methyl-butyl}-1H-imidazole-4-carboxylate
N-[3-(1-hydroxy-2-{3-[4-(4-N,N-dimethyl-sulphamoylamino-phenyl)-imidazol-1-yl]-1,1-dimethyl-propylamino}-ethyl)-phenyl]-benzenesulphonamide
N-(3-{2-[1,1-dimethyl-3-(2-oxo-3-pyridin-2-yl-imidazolidin-1-yl)-propylamino]-1-hydroxy-ethyl}-phenyl)-benzenesulphonamide
1-{3-[2-(3-benzenesulphonylamino-phenyl)-2-hydroxy-ethylamino]-3-methyl-butyl}-1H-imidazole-4-carboxylic acid
N-(3-{2-[1,1-dimethyl-3-(4-pyridin-4-yl-imidazol-1-yl)-propylamino]-1-hydroxy-ethyl}-phenyl)-benzenesulphonamide
benzyl 4-(1-{3-[2-(3-benzenesulphonylamino-phenyl)-2-hydroxy-ethylamino]-3-methyl-butyl}-1H-imidazol-4-yl)-benzoate
4-(1-{3-[2-(3-benzenesulphonylamino-phenyl)-2-hydroxy-ethylamino]-3-methyl-butyl}-1H-imidazol-4-yl)-benzoic acid
N-[3-(1-hydroxy-2-{3-[3-(4-hydroxy-phenyl)-2-oxo-imidazolidin-1-yl]-1,1-dimethyl-propylamino}-ethyl)-phenyl]-benzenesulphonamide
N-[4-(1-{3-[2-(3-benzenesulphonylamino-phenyl)-2-hydroxy-ethylamino]-3-methyl-butyl}-1H-imidazol-4-yl)-phenyl]-acetamide
N-[3-(2-{3-[4-(3,5-dimethyl-isoxazol-4-yl)-imidazol-1-yl]-1,1-dimethyl-propylamino}-1-hydroxy-ethyl)-phenyl]-benzenesulphonamide
N-[3-(1-hydroxy-2-{3-[4-(6-methoxy-pyridin-3-yl)-imidazol-1-yl]-1,1-dimethyl-propylamino}-ethyl)-phenyl]-benzenesulphonamide
N-[3-(2-{1,1-dimethyl-3-[4-(5-methyl-thiophen-2-yl)-imidazol-1-yl]-propylamino}-1-hydroxy-ethyl)-phenyl]-benzenesulphonamide
N-[3-(2-{3-[4-(4-fluoro-phenyl)-imidazol-1-yl]-1,1-dimethyl-propylamino}-1-hydroxy-ethyl)-phenyl]-benzenesulphonamide
N-(3-{2-[1,1-dimethyl-3-(5-nitro-benzimidazol-1-yl)-propylamino]-1-hydroxy-ethyl}-phenyl)-benzenesulphonamide
N-[3-(1-hydroxy-2-{3-[4-(4-methoxy-phenyl)-[1,2,3]triazol-1-yl]-1,1-dimethyl-propylamino}-ethyl)-phenyl]-benzenesulphonamide
N-(3-{2-[1,1-dimethyl-3-(4-thiophen-2-yl-imidazol-1-yl)-propylamino]-1-hydroxy-ethyl}-phenyl)-benzenesulphonamide
N-[3-(2-{3-[4-(4-dimethylamino-phenyl)-imidazol-1-yl]-1,1-dimethyl-propylamino}-1-hydroxy-ethyl)-phenyl]-benzenesulphonamide
ethyl 4-(1-{3-[2-(3-benzenesulphonylamino-phenyl)-2-hydroxy-ethylamino]-3-methyl-butyl}-1H-imidazol-4-yl)-benzoate
N-[3-(1-hydroxy-2-{3-[3-(4-methoxy-phenyl)-2-oxo-imidazolidin-1-yl]-1,1-dimethyl-propylamino}-ethyl)-phenyl]-benzenesulphonamide
N-[3-(2-{1,1-dimethyl-3-[3-(4-nitro-phenyl)-2-oxo-imidazolidin-1-yl]-propylamino}-1-hydroxy-ethyl)-phenyl]-benzenesulphonamide
N-[3-(1-hydroxy-2-{3-[3-(4-methoxy-phenyl)-5-methyl-[1,2,4]triazol-1-yl]-1,1-dimethyl-propylamino}-ethyl)-phenyl]-benzenesulphonamide
N-[3-(2-{3-[3-(4-amino-phenyl)-2-oxo-imidazolidin-1-yl]-1,1-dimethyl-propylamino}-1-hydroxy-ethyl)-phenyl]-benzenesulphonamide
N-[3-(2-{3-[4-(1-acetyl-2-amino-propenyl)-imidazol-1-yl]-1,1-dimethyl-propylamino}-1-hydroxy-ethyl)-phenyl]-benzenesulphonamide
N-(3-{2-[3-(5-amino-benzoimidazol-1-yl)-1,1-dimethyl-propylamino]-1-hydroxy-ethyl}-phenyl)-benzenesulphonamide
1-{3-[2-(3-benzenesulphonylamino-phenyl)-2-hydroxy-ethylamino]-3-methyl-butyl}-1H-benzimidazole-5-carboxylic acid
ethyl 1-{3-[2-(3-benzenesulphonylamino-phenyl)-2-hydroxy-ethylamino]-3-methyl-butyl}-1H-benzimidazole-5-carboxylate
N-{3-[2-(1,1-dimethyl-3-{4-[4-(1H-tetrazol-5-yl)-phenyl]-imidazol-1-yl}-propylamino)-1-hydroxy-ethyl]-phenyl}-benzenesulphonamide
4-(1-{3-[2-(3-benzenesulphonylamino-phenyl)-2-hydroxy-ethylamino]-3-methyl-butyl}-1H-imidazol-4-yl)-N-hydroxy-benzamide

By the term alkyl groups, as well as alkyl groups which are part of other groups, are meant, unless stated otherwise, branched and unbranched alkyl groups with 1 to 10 carbon atoms, while groups with 1 to 6 carbon atoms are preferred. Particularly preferred are alkyl groups with 1 to 4 carbon atoms, particularly those with 1 or 2 carbon atoms. The following are mentioned by way of example: methyl ethyl, propyl, butyl, pentyl, hexyl, octyl, nonyl and decyl. Unless stated otherwise, the above-mentioned terms propyl, butyl, pentyl, hexyl, octyl, nonyl and decyl include all the possible isomeric forms. For example, the term propyl includes the two isomeric groups n-propyl and iso-propyl, the term butyl includes n-butyl, iso-butyl, sec. butyl and tert.-butyl, the term pentyl includes iso-pentyl, neo-pentyl etc. In the above-mentioned alkyl groups one or more hydrogen atoms may optionally be replaced by other groups. For example these alkyl groups may be substituted by the halogen atoms fluorine, chlorine, bromine or iodine. The substituents are preferably fluorine or chlorine. Particularly preferred is the substituent fluorine. It is also possible for all the hydrogen atoms of the alkyl group to be replaced. Similarly, in the above-mentioned alkyl groups, unless otherwise described, one or more hydrogen atoms may optionally be replaced for example by OH, NO₂, CN or an optionally substituted group selected from among —O—(C₁-C₅-alkyl), preferably methoxy or ethoxy, —O—(C₆-C₁₄-aryl), preferably phenyloxy, —O-heteroaryl, preferably —O-thienyl, —O-thiazolyl, —O-imidazolyl, —O-pyridyl, —O-pyrimidyl or —O-pyrazinyl, saturated or unsaturated —O-heterocycloalkyl, preferably —O-pyrazolyl, —O-pyrrolidinyl, —O-piperidinyl, —O-piperazinyl or —O-tetrahydro-oxazinyl, C₆-C₁₄-aryl, preferably phenyl, heteroaryl, preferably thienyl, thiazolyl, imidazolyl, pyridyl, pyrimidyl or pyrazinyl, saturated or unsaturated heterocycloalkyl, preferably pyrazolyl, pyrrolidinyl, piperidinyl, piperazinyl or tetrahydro-oxazinyl, an amine group, preferably methylamine, benzylamine, phenylamine or heteroarylamine, saturated or unsaturated bicyclic ring systems, preferably benzimidazolyl and C₃-C₈-cycloalkyl, preferably cyclohexyl or cyclopropyl.

Examples of alkenyl groups as well as alkenyl groups which are part of other groups include branched and unbranched alkyl groups with 1 to 10 carbon atoms, preferably 1 to 6, particularly preferably 1 to 4 carbon atoms, which contain at least one carbon-carbon double bond. Examples include: ethenyl, propenyl, methylpropenyl, butenyl, pentenyl, hexenyl, heptenyl, methylheptenyl, octenyl, nonenyl and decenyl. Unless stated otherwise, the above-mentioned terms propenyl, butenyl, pentenyl, hexenyl, heptenyl, octenyl, nonenyl and decenyl include all the possible isomeric forms. For example the term butenyl includes the isomeric groups but-1-enyl, but-2-enyl and but-3-enyl, etc.

In the above-mentioned alkenyl groups one or more hydrogen atoms may optionally be replaced by other groups. For example, these alkenyl groups may be substituted by the halogen atoms fluorine, chlorine, bromine or iodine. The substituents fluorine or chlorine are preferred. Particularly preferred is the substituent fluorine. It is also possible for all the hydrogen atoms of the alkenyl group to be replaced.

Examples of alkynyl groups as well as alkynyl groups which are part of other groups include branched and unbranched alkyl groups with 1 to 10 carbon atoms, preferably 1 to 6, particularly preferably 1 to 4 carbon atoms, which contain at least one carbon-carbon triple bond. Examples include: ethynyl, propynyl, butynyl, pentynyl, hexynyl, heptynyl, octynyl, nonynyl and decynyl. Unless otherwise stated, the above-mentioned terms propynyl, butynyl, pentynyl, hexynyl, heptynyl, octynyl, nonynyl and decynyl include all the possible isomeric forms. For example the term butynyl includes the isomeric groups but-1-ynyl, but-2-ynyl and but-3-ynyl, etc. In the above-mentioned alkynyl groups one or more hydrogen atoms may optionally be replaced by other groups. For example, these alkynyl groups may be substituted by the halogen atoms fluorine, chlorine, bromine or iodine. The substituents fluorine or chlorine are preferred. Particularly preferred is the substituent fluorine. It is also possible for all the hydrogen atoms of the alkynyl group to be replaced.

The term aryl denotes an aromatic ring system with 6 to 18 carbon atoms, preferably 6 to 14 carbon atoms, preferably 6 or 10 carbon atoms, particularly preferably phenyl, which may optionally be substituted and may preferably carry one or more of the following substituents: OH, NO₂, CN, —OCHF₂, —OCF₃, —NH₂, —NH-alkyl, —N(alkyl)-alkyl, —NH-aryl, —N(alkyl)-aryl, —NHCO-alkyl, —NHCO-aryl, —N(alkyl)-CO-alkyl, —N(alkyl)-CO aryl, —NHSO₂-alkyl, —NHSO₂—N(alkyl)₂, —NHSO₂-aryl, —N(alkyl)-S0₂-alkyl, —N(alkyl)-SO₂-aryl, CO₂-alkyl, SO₂-alkyl, SO₂-aryl, —CONH(OH), —CONH-alkyl, —CONH-aryl, —CON(alkyl)-alkyl, —CON(alkyl)-aryl, —SO₂NH-alkyl, —SO₂NH-aryl, —SO₂N(alkyl)-alkyl, SO₂N(alkyl)-aryl, —O-alkyl, —O-aryl —S-alkyl, —S-aryl, tetrazolyl, halogen, for example fluorine, chlorine, bromine or iodine, preferably fluorine or chlorine, particularly fluorine, C₁-C₁₀-alkyl, preferably C₁-C₅-alkyl, particularly preferably C₁-C₃-alkyl, most particularly preferably methyl or ethyl, —O—(C₁-C₃-alkyl) preferably methoxy or ethoxy, —COOH or —CONH₂.

By heteroaryl groups are meant 5- to 10-membered mono- or bicyclic heteroaryl rings, wherein one to three carbon atoms may in each case be replaced by a heteroatom selected from among oxygen, nitrogen or sulphur. Examples are furan, thiophene, pyrrole, pyrazole, imidazole, triazole, tetrazole, pyridine, pyridazine, pyrimidine, pyrazine, triazine, oxazole, isoxazole, thiazole, thiadiazole, oxadiazole, while each of the above-mentioned heterocycles may optionally also be annellated to a benzene ring, such as for example benzimidazole, and these heterocycles may optionally be substituted and may preferably carry one or more of the following substituents. OH, NO₂, CN, —NH₂, —NH-alkyl, —N(alkyl)-alkyl, —NH-aryl, —N(alkyl)-aryl, —NHCO-alkyl, —NHCO-aryl, —N(alkyl)-CO-alkyl, —N (alkyl)-CO-aryl, —NHSO₂-alkyl, —NHSO₂-aryl, —N(alkyl)-SO₂-alkyl, —N(alkyl)-SO₂-aryl, —CO₂-alkyl, —SO₂-alkyl, —SO₂-aryl, —CONH-alkyl, —CONH-aryl, —CON(alkyl)-alkyl, —CON(alkyl)-aryl, —SO₂NH-alkyl, —SO₂NH-aryl, —SO₂N(alkyl)-alkyl, —SO₂N(alkyl)-aryl, —O-alkyl, —O-aryl —S-alkyl, —S-aryl, —CONH₂, halogen, preferably fluorine or chlorine, C₁-C₁₀-alkyl, preferably C₁-C₅-alkyl, preferably C₁-C₃-alkyl, particularly preferably methyl or ethyl, —O—(C₁-C₃-alkyl) preferably methoxy or ethoxy, —COOH, —COOCH₃, —CONH₂, SO-alkyl, —SO₂-alkyl, —SO₂H, —SO₃-alkyl or optionally substituted phenyl.

The term cycloalkyl groups denotes saturated or unsaturated cycloalkyl groups with 3 to 8 carbon atoms such as for example cyclopropyl, cyclobutyl, cyclopentyl, cyclopentenyl, cyclohexyl, cyclohexenyl, cycloheptyl or cyclooctyl, preferably cyclopropyl, cyclopentyl or cyclohexyl, while each of the above-mentioned cycloalkyl groups may optionally also carry one or more substituents or be annellated to a benzene ring.

The terms heterocycloalkyl or heterocyclyl groups, unless otherwise described in the definitions, denote 5-, 6- or 7-membered, saturated or unsaturated heterocycles which may contain as heteroatoms nitrogen, oxygen or sulphur, such as for example tetrahydrofuran, tetrahydrofuranone, γ-butyrolactone, α-pyran, γ-pyran, dioxolane, tetrahydropyran, dioxane, dihydrothiophene, thiolane, dithiolane, pyrroline, pyrrolidine, pyrazoline, pyrazolidine, imidazoline, imidazolidine, tetrazole, piperidine, pyridazine, pyrimidine, pyrazine, piperazine, triazine, tetrazine, morpholine, thiomorpholine, diazepan, oxazine, tetrahydro-oxazinyl, isothiazole, pyrazolidine, preferably pyrazolyl, pyrrolidinyl, piperidinyl, piperazinyl or tetrahydro-oxazinyl, while the heterocyclic group may optionally be substituted.

The compounds of the above general formula (Ib) which contain a group that can be cleaved in-vivo are so-called prodrugs, and compounds of general formula (Ib) which contain two groups that can be cleaved in-vivo are so-called double prodrugs.

By a group that can be converted in-vivo into a carboxy group is meant for example an ester of formula —CO₂R¹¹, wherein

R¹¹ denotes hydroxymethyl, alkenyl, alkynyl, aryl, heteroaryl, cycloalkenyl, heterocyclo-alkyl, C₁-C₃ alkoxycarbonyl, 1,3-dihydro-3-oxo-1-isobenzofuranol, —C(-alkyl)(-alkyl)-OC(O)-alkyl, —CHC(O)NH(-Alkyl), —CHC(O)N(-alkyl)(-alkyl), -alkyl, preferably C₁-C₆-alkyl, particularly preferably methyl, ethyl, n-propyl, iso-propyl, n-butyl, n-pentyl or n-hexyl,
cycloalkyl, preferably C₁-C₆-cycloalkyl, particularly preferably cyclohexyl, —(C₁-C₃-alkyl)-aryl, preferably (C₁-C₃-alkyl)-phenyl, particularly preferably benzyl, —CHC(O)N(-alkyl)(-alkyl), preferably —CHC(O)N(—C₁-C₃-alkyl)(—C₁-C₃-alkyl), particularly preferably —CHC(O)N(CH₃)₂,
—CH(-alkyl)OC(O)-alkyl, preferably —CH(—CH₃)OC(O)(—C₁-C₆-alkyl), particularly preferably —CH(—CH₃)OC(O)-methyl, —CH(—CH₃)OC(O)-ethyl, —CH(—CH₃)OC(O)-n-propyl, —CH(—CH₃)OC(O)-n-butyl or —CH(—CH₃)OC(O)-t-butyl, or
—CH₂OC(O)-alkyl, preferably —CH₂OC(O)(—C₁-C₆-alkyl), particularly preferably —CH₂OC(O)-methyl, —CH₂OC(O)-ethyl, —CH₂OC(O)-n-propyl, —CH₂OC(O)-n-butyl or —CH₂OC(O)-t-butyl.

By a group that can be converted in-vivo into a sulphonamide or amino group is meant for example one of the following groups:

—OH, -formyl, —C(O)-alkyl, —C(O)-aryl, —C(O)-heteroaryl, —CH₂OC(O)-alkyl, —CH(-alkyl)OC(O)-alkyl, —C(-alkyl)(-alkyl)OC(O)-alkyl,
—CO₂-alkyl, preferably C₁-C₉-alkoxy-carbonyl, particularly preferably methoxy-carbonyl, ethoxycarbonyl, n-propyloxycarbonyl, isopropyloxycarbonyl, n-butyl-oxycarbonyl, n-pentyloxycarbonyl, n-hexyloxycarbonyl, cyclohexyloxycar-bonyl, n-heptyloxycarbonyl, n-octyloxycarbonyl or n-nonyloxycarbonyl, —CO₂(C₁-C₃-alkyl)-aryl, preferably —CO₂(C₁-C₃-alkyl)-phenyl, particularly preferably benzyloxycarbonyl,
—C(O)-aryl, preferably benzoyl,
—C(O)-heteroaryl, preferably pyridinoyl or nicotinoyl or —C(O)-alkyl, preferably —C(O)(—C₁-C₆-alkyl), particularly preferably 2-methyl-sulphonylethoxycarbonyl, 2-(2-ethoxy)-ethoxycarbonyl.

The halogen is generally fluorine, chlorine, bromine or iodine, preferably chlorine or fluorine, particularly preferably fluorine.

The compounds according to the invention may be present in the form of the individual optical isomers, mixtures of the individual enantiomers, diastereomers or racemates, prodrugs and double prodrugs and in the form of the tautomers, salts, solvates and hydrates, as well as in the form of the free bases or the corresponding acid addition salts with pharmacologically acceptable acids—such as for example acid addition salts with hydrohalic acids, for example hydrochloric or hydrobromic acid, or organic acids, such as for example oxalic, fumaric, diglycolic, formic, malic, benzoic, benzenesulphonic, camphorsulphonic, acetic, ethanesulphonic, glutamic, maleic, mandelic, lactic, phosphoric, nitric, sulphuric, succinic, para-toluenesulphonic, trifluoroacetic, tartaric, citric or methanesulphonic acid.

If desired, the new compounds of formula (Ia) or (Ib) thus obtained, if they contain a carboxy group or another acid group, may subsequently be converted into the salts thereof with inorganic or organic bases, particularly for pharmaceutical use into the physiologically acceptable salts thereof. Bases which may be used include for example sodium hydroxide, potassium hydroxide, cyclohexylamine, ethanolamine, diethanolamine and triethanolamine.

Moreover the compounds of general formula (Ia) or (Ib) obtained may be resolved into their enantiomers and/or diastereomers.

Thus, for example, the compounds of general formula (Ib) obtained which occur as stereoisomers may be resolved into their optical antipodes according to WO 05/108373.

The substituent R¹ may be optionally substituted aryl or heteroaryl, preferably substituted phenyl. Particularly preferably the substituent R¹ denotes phenyl.

The substituent R² may be a heteroaryl or heterocyclyl mono- or polysubstituted by R¹⁰, where R² contains at least one nitrogen atom. Particularly preferred is a triazole mono- or polysubstituted by R¹⁰, a 1,4-dioxo-3,4-dihydro-1H-phthalazine mono- or polysubstituted by R¹⁰, a 2-oximidazolidine mono- or polysubstituted by R¹⁰, a benzimidazole mono- or polysubstituted by R¹⁰ or an imidazole mono- or polysubstituted by R¹⁰.

Most particularly preferred meanings of the substituent R² are a 1H-[1,2,3]triazol-1-yl monosubstituted by R¹⁰, a 1,4-dioxo-3,4-dihydro-1H-phthalazin-2-yl monosubstituted by R¹⁰, a 2-oxo-imidazolidin-1-yl monosubstituted by R¹⁰, a benzimidazol-1-yl monosubstituted by R¹⁰ or an imidazol-1-yl monosubstituted by R¹⁰.

The substituents R³ and R⁴ may independently of one another denote hydrogen or an optionally substituted group selected from among C₃-C₆-cycloalkyl or C₁-C₅-alkyl, preferably C₁-C₅-alkyl, or

R³ and R⁴ together denote a 2- to 7-membered alkylene bridge, preferably a 2- to 5-membered alkylene bridge, particularly an ethylene bridge.

A substituted R³ or R⁴ is preferably substituted by C₁-C₃-alkyl.

Preferably R³ denotes methyl.

Preferably R⁴ denotes methyl.

The substituents R⁵, R⁶ and R⁷ may independently of one another denote hydrogen or a group selected from among halogen, cyano, —NR⁸CO—(C₁-C₅-alkyl), —NR⁸CO-aryl, —NR⁸SO₂—(C₁-C₅-alkyl), NR⁸SO₂-aryl, —CO₂R⁸, —SO₂R⁸, —CONHR⁸, —SO₂NHR⁸, —OR⁸, optionally substituted C₃-C₆-cycloalkyl and optionally substituted C₁-C₁₀-alkyl, preferably hydrogen, halogen, cyano, methoxy, methanesulphonylamino, methanesulphonyl, difluoromethoxy, trifluoromethoxy, difluoromethyl or trifluoromethyl, particularly hydrogen, fluorine, chlorine or cyano.

A most particularly preferred meaning of the substituents R⁵, R⁶ and R⁷ is hydrogen.

The substituent R⁸ may denote hydrogen or C₁-C₅-alkyl, preferably methyl.

The substituent R⁹ may represent hydrogen, optionally substituted aryl or optionally substituted heteroaryl, preferably optionally substituted phenyl, pyridyl or thiophenyl.

The substituent R¹⁰ may represent OH, NO₂, CN, —OCHF₂, —OCF₃, —NH₂, —NH-alkyl, —N(-alkyl)alkyl, —NH-aryl, —N(-alkyl)-aryl, —NHCO-alkyl, —NHCO-aryl, —N(-alkyl)CO-alkyl, —N(-alkyl)CO-aryl, —NHSO₂-alkyl, —NHSO₂-aryl, —N(-alkyl)SO₂-alkyl, —N(-alkyl)SO₂-aryl-CO₂-alkyl, —SO₂-alkyl, —SO₂-aryl, —CONH-alkyl, —CONH-aryl, —CON(-alkyl)-alkyl, —CON(-Alkyl)-aryl, —SO₂NH-alkyl, —SO₂NH-aryl, —SO₂N(-alkyl)-alkyl, —SO₂N(-alkyl)-aryl, —O-alkyl, —O-aryl, —S-alkyl, —S-aryl, halogen, C₁-C₁₀-alkyl, —O—(C₁-C₃-alkyl), —COOH, —CONH₂, —CON(-alkyl)SO₂-alkyl, —CONHSO₂-alkyl, —CONHOH, 2,5-dihydro-5-oxo-4H-1,2,4-oxadiazol-3-yl, 2,5-dihydro-5-oxo-4H-1,2,4-thiadiazol-3-yl, 2,5-dihydro-2-oxo-3H-1,2,4,5-oxathiadiazol-4-yl, tetrazolyl, heterocyclyl, aryl or heteroaryl.

Preferably R¹⁰ denotes —OH, —NO₂, —CN, —NH₂, —I, —N(CH₃)₂, —NHCO₂CH₃, —NHSO₂CH₃, —SO₂N(CH₃)₂, —CO₂H, benzyloxycarbonyl, ethoxycarbonyl, methoxycarbonyl, —CONHOH, tetrazol-5-yl, pyridin-4-yl, pyridin-2-yl, 6-methoxy-pyridin-3-yl, phenyl, 4-hydroxyphenyl, 4-fluorophenyl, 4-methoxy-phenyl, 4-aminophenyl, 4-nitrophenyl, thiophen-2-yl, 5-methyl-thiophen-2-yl, 3,5-dimethyl-isoxazol-4-yl or 1-acetyl-2-amino-propenyl.

The preparation of the compounds of formula (Ib) according to the invention may be taken from WO 05/108373.

In the use according to the invention the specified compounds are administered to a patient in an effective amount.

As has been found, the compounds of general formula (Ia) and (Ib) are characterised by their great versatility in the therapeutic field. Particular mention should be made of those applications in which the effects of beta-3-agonists, particularly selective beta-3-agonists play a part.

Such diseases include for example:

urge incontinence, stress incontinence, mixed incontinence, overactive bladder (OAB) in the forms of wet OAB or dry OAB, OAB with imperative need to urinate, with or without urge incontinence, with or without increased frequency of urination, with or without nocturnal urination, dysuria, nycturia, pollacisuria, build-up of residual urine. Of these indications, OAB with increased frequency of urination, with or without urge incontinence, with or without nocturnal urination, is preferred.

The compounds may also be used in cases of pain in the prostate or of the lower urogenital tract. The diseases in question include benign prostatic hyperplasia (BPH), prostatitis, particularly chronic abacterial prostatitis, of neurogenic, muscular or bacterial origin, chronic pain syndrome of the pelvis, pelvic myoneuropathy, prostatodynia, LUTS (lower urinary tract symptoms), obstructive bladder emptying disorders (BOO) and/or prostatopathy.

The use according to the invention is directed not only to causative treatment of the above indications, but also to the treatment of the accompanying symptoms, particularly any related pain or problems of urine release, pain and discomfort in the region of the prostate or the lower urinary tract including the penis, pain during erection or ejaculation, pain on defecation, erectile disorders.

The compounds are also suitable for the treatment of irritable bowel syndrome, particularly irritable bowel syndrome with prevalent diarrhoea.

In the use according to the invention the specified compounds are administered to a patient in an effective amount.

Details of formulation examples and details of formulation ingredients may be taken from WO 2004/039784 and WO 05/108373.

The new compounds may be used for the prevention, short- or long-term treatment of the above-mentioned diseases, also in combination with other active substances which are used for the same indications.

1. A method for treating overactive bladder which comprises administering, to a host suffering from overactive bladder, a therapeutically effective amount of a compound of the formula (Ia) 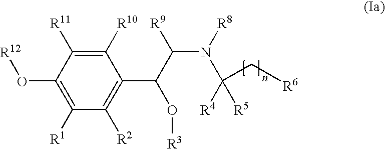
wherein
R¹, R², R¹⁰, R¹¹ independently of one another denote a group selected from among hydrogen, halogen, CN, NO₂, and —NHCXNH₂ or
a group selected from among optionally substituted —COR⁷, —COOR⁷, —CONR⁷R¹³, —OR¹⁴, NR¹³R¹⁵, C₁-C₁₀-alkyl, C₃-C₈-cycloalkyl, —NR¹⁶CX—R¹⁷, —NR¹⁸CX—OR¹⁹, —NR²⁰SOₘR²¹, —SOₚNR²²R²³ and —SO_{q}R²⁴.
m, p, q denotes 0, 1 or 2
n denotes 0, 1, 2 or 3
R³ denotes hydrogen or a group selected from among optionally substituted C₁-C₁₀-alkyl, C₆-C₁₀-aryl, heterocyclyl and C₃-C₈-cycloalkyl, —CX—C₁-C₁₀-alkyl, —CX—C₆-C₁₄-aryl,
R⁴, R⁵ independently of one another denote hydrogen, halogen or optionally substituted C₁-C₁₀-alkyl,
or
R⁴ and R⁵ together denote a C₃-C₈-alkyl bridge,
R⁶ denotes a group selected from among the general formulae 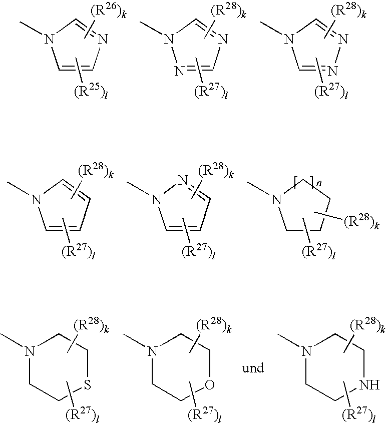
l,k independently of one another denote 1, 2 or 3,
R²⁵, R²⁶, R²⁷, R²⁸ independently of one another denote a group selected from among hydrogen, OH, halogen, CN and NO₂,
or
a group selected from among optionally substituted C₁-C₁₀-alkyl, C₆-C₁₈-aryl, heteroaryl, heterocyclyl, —CX—R¹⁷, —OR¹⁴, NR¹³R¹⁵, C₂-C₈-cycloalkyl —NR²⁰SOₘR²¹, SOₚNR²²R²³, —SO_{q}R²⁴—NR¹⁸CX—R¹⁹, —NR²⁰CXOR¹⁷, wherein R²⁵ and R²⁶ cannot simultaneously represent hydrogen,
R⁸ denotes hydrogen or a group selected from among optionally substituted C₁-C₁₀-alkyl, C₆-C₁₈-aryl, —SO_{q}—C₁-C₁₀-alkyl, —SO_{q}—C₆-C₁₄-aryl, —CX—C₁-C₁₀-alkyl, —CX—C₆-C₁₄-aryl, C₆-C₁₀-aryl, heterocyclyl and C₃-C₈-cycloalkyl
R⁹ denotes hydrogen or a group selected from among optionally substituted C₁-C₁₀-alkyl, C₆-C₁₄-aryl, heteroaryl, C₃-C₈-cycloalkyl and heterocycloalkyl,
R¹² denotes hydrogen or a group selected from among optionally substituted benzyl, C₁-C₁₂-alkyl and C₆-C₁₄-aryl,
R⁷, R¹³, R¹⁵, R¹⁶, R¹⁸, R²⁰, R²², R²³ independently of one another denote hydrogen, or
a group selected from among optionally substituted C₁-C₁₀-alkyl, C₆-C₁₄-aryl, heterocyclyl and C₃-C₈-cycloalkyl
R¹⁴, R¹⁹, R²⁹ independently of one another denote hydrogen or a group selected from among optionally substituted C₁-C₁₀-alkyl, C₆-C₁₄-aryl, C₃-C₈-cycloalkyl, heteroaryl, heterocyclyl, —CXNR₁₃R₁₅ and —CXR₇
R¹⁷ denotes a group selected from among C₁-C₁₀-alkyl, C₆-C₁₄-aryl, heterocyclyl, heteroaryl and C₃-C₈-cycloalkyl
R²¹, R²⁴ independently denote hydrogen or OH, or
a group selected from among optionally substituted N(C₁-C₁₀-alkyl)₂, N(C₃-C₈-cycloalkyl), C₁-C₁₀-alkyl, C₆-C₁₄-aryl, heterocyclyl, heteroaryl and C₃-C₈-cycloalkyl
and
X denotes O, S or NR²⁹,
or a pharmacologically acceptable salt thereof,
or a compound of the formula (Ib) 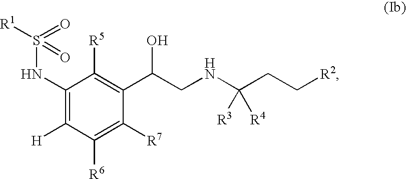
wherein
R¹ denotes an optionally substituted aryl or heteroaryl group,
R² denotes an optionally substituted heteroaryl or heterocyclyl group, where R² contains at least one nitrogen atom,
R³ and R⁴ independently of one another denote a hydrogen atom or an optionally substituted group selected from among C₁-C₅-alkyl, C₃-C₆-cycloalkyl, heterocyclyl, aryl and heteroaryl, or
R³ and R⁴ together denote a 2- to 7-membered alkylene bridge,
R⁵, R⁶ and R⁷ independently of one another denote a hydrogen atom or a group selected from among optionally substituted C₁-C₁₀-alkyl, alkenyl, alkynyl, C₆-C₁₀-aryl, heterocyclyl, C₃-C₈-cycloalkyl, —NR⁸—C₁-C₅-alkyl, —NR⁸-aryl, halogen, CN, —NR⁸CO—(C₁-C₅-alkyl), —NR⁸CO-aryl, —NR⁸SO₂—(C₁-C₅-alkyl), —NR⁸SO₂-aryl, —CO₂R⁸, —SO₂R⁸, —CONHR⁸, —SO₂NHR⁸ and —OR⁸, while the above-mentioned alkyl groups may each be substituted, and
R⁸ denotes a hydrogen atom or a C₁-C₅-alkyl group,
or a pharmacologically acceptable salt thereof.

2. The method according to claim 1, wherein, in the compound of formula (Ia):
R¹⁰, R¹¹ independently of one another denote hydrogen or halogen,
m, p, q independently of one another denote 0, 1 or 2
n denotes 0, 1, 2 or 3
R³ denotes hydrogen or C₁-C₅-alkyl
R⁴, R⁵ independently of one another denote hydrogen or C₁-C₅-alkyl,
R⁸ denotes a group selected from among hydrogen, C₁-C₅-alkyl, —SO_{q}—C₁-C₅-alkyl, —SO_{q}—C₆-C₁₄-aryl, phenyl and C₃-C₆-cycloalkyl
R⁹ denotes hydrogen or C₁-C₁₀-alkyl
R¹² denotes hydrogen or benzyl
R¹³, R¹⁵, R¹⁶, R¹⁸ independently of one another denote a group selected from among hydrogen, C₁-C₅-alkyl, C₃-C₆-cycloalkyl and phenyl
R¹⁴, R¹⁹ independently of one another denote hydrogen or C₁-C₅-alkyl,
and
R¹⁷ denotes optionally substituted C₁-C₅-alkyl or C₆-C₁₀-aryl.

3. The method of claim 1, wherein in the compound of formula (Ia):
R¹⁰, R¹¹ denotes hydrogen
m, p, q denotes 0, 1 or 2
n denotes 0, 1, 2 or 3
R³ denotes hydrogen
R⁴, R⁵ independently of one another denote hydrogen or methyl,
R⁸ denotes hydrogen, —SO_{q}—C₆-C₁₄-aryl or —SO₂—C₁-C₅-alkyl
R⁹ denotes hydrogen
R¹² denotes hydrogen or benzyl,
R¹³, R¹⁵, R¹⁶, R¹⁸ independently of one another denote a group selected from among hydrogen, C₁-C₁₅-alkyl and phenyl,
R¹⁴, R¹⁹ independently of one another denote hydrogen or C₁-C₅-alkyl,
and
R¹⁷ denotes C₁-C₅-alkyl or C₆-C₁₄-aryl.

4. The method of claim 1, wherein in the compound of formula (Ia):
R¹ denotes a group selected from among hydrogen, NO₂, NH₂, —NHCX—R¹⁷ and —NHSO₂R²¹.
R² denotes hydrogen or halogen
n denotes 2,
R³ denotes hydrogen
R⁴, R⁵ denotes hydrogen or methyl
R⁶ denotes a group selected from among the general formulae 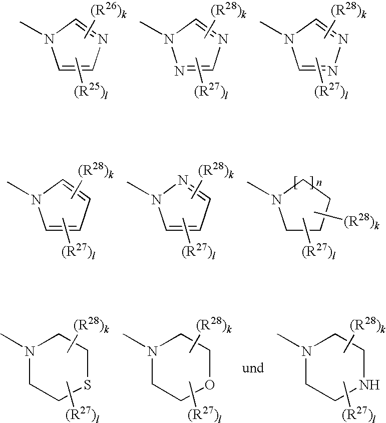
l,k denote 1
R²⁶, R²⁷ denote hydrogen,
R⁸ denotes hydrogen or —SO₂CH₃,
R⁹ denotes hydrogen,
R¹⁰, R¹¹ denotes hydrogen, and
R¹² denotes hydrogen or benzyl.

5. The method of claim 1, wherein in the compound of formula (Ia):
R⁶ denotes a group selected from among the general formulae

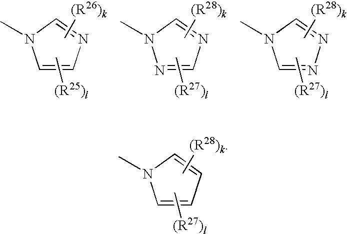

6. The method of claim 1 wherein, in the compound of formula (Ia):
R⁶ denotes an optionally substituted group of formula (j)

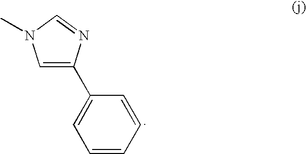

7. The method of claim 1 wherein, in the compound of formula (Ib):
R² to R⁷ are defined as in claim 1, and
R¹ denotes an optionally substituted phenyl group,
or a salt thereof.

8. The method of claim 1 wherein, in the compound of formula (Ib):
R¹ and R³ to R⁷ are defined as in claim 1, and
R² denotes a group selected from among the optionally substituted groups of formulae: 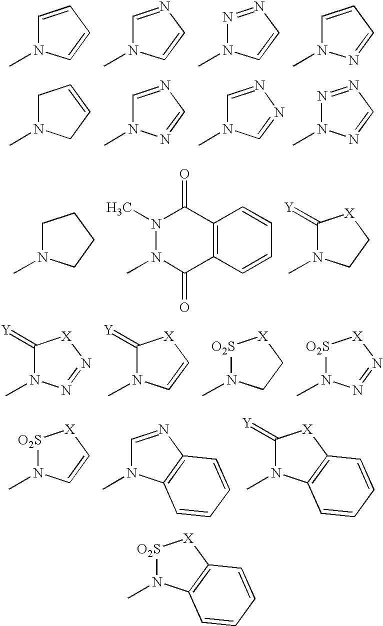
wherein the above-mentioned groups may each be substituted by one or more groups R¹⁰ and
R¹⁰ denotes OH, NO₂, CN, —OCHF₂, —OCF₃, —NH₂, —NH-alkyl, —N(-alkyl)-alkyl, —NH-aryl, —N(-alkyl)-aryl, —NHCO-alkyl, —NHCO₂-alkyl, —NHCO-aryl, —N(-alkyl)-CO-alkyl, —N(-alkyl)-CO-aryl, —NHSO₂-alkyl, —NHSO₂-aryl, —N(-alkyl)-SO₂-alkyl,
—N(-alkyl)-SO₂-aryl, —CO₂-alkyl, —SO₂-alkyl, —SO₂-aryl, —CONH-alkyl, —CONH-aryl, —CON(-alkyl)-alkyl, —CON(-alkyl)-aryl, —SO₂NH-alkyl, —SO₂NH-aryl,
—SO₂N(-alkyl)-alkyl, —SO₂N(-alkyl)-aryl, —O-alkyl, —O-aryl, —S-alkyl, —S-aryl, halogen, C₁-C₁₀-alkyl, —O—(C₁-C₃-alkyl), —COOH, —CONH₂, —CON(-alkyl)-SO₂-alkyl, —CONHSO₂-alkyl, —CONHOH, 2,5-dihydro-5-oxo-4H-1,2,4-oxadiazol-3-yl, 2,5-dihydro-5-oxo-4H-1,2,4-thiadiazol-3-yl, 2,5-dihydro-2-oxo-3H-1,2,4,5-oxathiadiazol-4-yl, 1-acetyl-2-amino-propen-1-yl, tetrazolyl, heterocyclyl, aryl or heteroaryl,
and wherein X denotes an oxygen atom or an —NR⁹— group and
Y denotes an oxygen or sulphur atom,
and R⁹ denotes a hydrogen atom or a group selected from among C₁-C₁₀-alkyl, C₃-C₈-cycloalkyl, heterocyclyl, aryl or heteroaryl, while the groups mentioned for R⁹ hereinbefore may each be substituted by one of the groups mentioned for R¹⁰,
or a salt thereof.

9. The method of claim 1 wherein, in the compound of formula (Ib):
R³ and R⁴ independently of one another denote a hydrogen atom or a methyl or ethyl group
or
R³ and R⁴ together denote a 2- to 5-membered alkylene bridge,
or a salt thereof.

10. The method of claim 1 wherein, in the compound of formula (Ib):
R⁵, R⁶ and R⁷ independently of one another denote hydrogen, optionally substituted C₁-C₁₀-alkyl, halogen, CN, —NR⁸CO—(C₁-C₅-alkyl), —NR⁸SO₂—(C₁-C₅-alkyl), —CO₂R⁸, —SO₂R⁸, —CONHR⁸, —SO₂NHR⁸ or —OR⁸ and
R⁸ denotes a hydrogen atom or a C₁-C₅-alkyl group,
or a salt thereof.

11. The method of claim 1 wherein, in the compound of formula (Ib):
R¹ denotes a phenyl group optionally substituted by a halogen atom or a cyano or nitro group,
R² denotes a group selected from among the optionally substituted groups of formulae 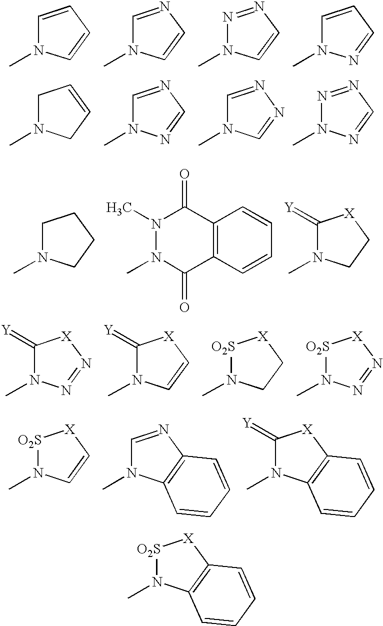
wherein the above-mentioned groups may each be substituted by one or more groups R¹⁰ and
R¹⁰ denotes OH, NO₂, CN, —OCHF₂, —OCF₃, —NH₂, —NH-alkyl, —N(-alkyl)-alkyl, —NH-aryl, —N(-alkyl)-aryl, —NHCO-alkyl, —NHCO₂-alkyl, —NHCO-aryl, —N(-alkyl)CO-alkyl, —N(-alkyl)-CO-aryl, —NHSO₂-alkyl, —NHSO₂-aryl, —N(-alkyl)-SO₂-alkyl, —N(-alkyl)-SO₂-aryl, —CO₂-alkyl, —SO₂-alkyl, —SO₂-aryl, —CONH-alkyl, —CONH-aryl, —CON(alkyl)-alkyl, —CON(-alkyl)-aryl, —SO₂NH-alkyl, —SO₂NH-aryl,
—SO₂N(-alkyl)-alkyl, —SO₂N(-alkyl)-aryl, —O-aryl, —S-alkyl, —S-aryl, halogen,
C₁-C₁₀-alkyl, —O—(C₁-C₃-alkyl), —COOH, —CONH₂, —CON(-alkyl)-SO₂-alkyl,
—CONHSO₂-alkyl, —CONHOH, 2,5-dihydro-5-oxo-4H-1,2,4-oxadiazol-3-yl, 2,5-dihydro-5-oxo-4H-1,2,4-thiadiazol-3-yl, 2,5-dihydro-2-oxo-3H-1,2,4,5-oxathiadiazol-4-yl, 1-acetyl-2-amino-propen-1-yl, tetrazolyl, heterocyclyl, aryl or heteroaryl,
and wherein X denotes an oxygen atom or an —NR⁹-group and
Y denotes an oxygen or sulphur atom,
R³ and R⁴ independently of one another each denote a methyl or ethyl group or
R³ and R⁴ together denote an ethylene bridge,
R⁵, R⁶ and R⁷ independently of one another each denote a hydrogen, fluorine or chlorine atom or a cyano, methoxy, methanesulphonylamino, methanesulphonyl, difluoromethoxy, trifluoromethoxy, difluoromethyl or trifluoromethyl group,
R⁹ denotes a hydrogen atom or an optionally substituted aryl or optionally substituted heteroaryl group,
or a salt thereof.

12. The method of claim 1 wherein the compound of formula (Ia) or (Ib) is the (R)-enantiomer.
